(19) United States
(12) Patent Application Publication
(10) Pub. No.: US 2016/0145668 A1
(43) Pub. Date: May 26, 2016

(54) UNCOUPLED CELL CULTURE METHOD

(71) Applicant: FERMENTALG, Libourne (FR)

(72) Inventors: Patrice GARNIER, LAGORCE (FR); Julien PAGLIARDINI, BORDEAUX (FR); Pierre CALLEJA, LIBOURNE (FR)

(21) Appl. No.: 14/904,381

(22) PCT Filed: Jul. 15, 2014
(86) PCT No.: PCT/FR2014/051809

§ 371 Date: Jan. 11, 2016

(30) Foreign Application Priority Data
Jul. 12, 2013 (FR) 1356918

(51) Int. Cl. C12P 23/00; C12P 7/64

(52) U.S. Cl. CPC C12P 23/00; C12P 7/6427

(57) ABSTRACT

Disclosed is a method for producing biomass by culturing cells in mixotrophic conditions, in particular in the presence of discontinuous and/or variable lighting conditions, and/or in heterotrophic conditions, making it possible to obtain an increase in both the cellular concentration and the production of molecules of interest.

The invention relates to a method for the production of biomass by cell culture under mixotrophic conditions, in particular in the presence of discontinuous and/or variable illumination with light, and/or under heterotrophic conditions making it possible to obtain both an increase in cell concentration and in the production of molecules of interest.

The method for the production of biomass according to the invention comprises the culture of the cells in continuous mode under mixotrophic conditions or under heterotrophic conditions in a fermenter, then the continuous feeding into fermenters operating in semi-continuous mode with the cells produced and their culture under mixotrophic conditions or under heterotrophic conditions in said fermenters.

The method is applicable to cells and, in particular, to eucaryotic or procaryotic unicellular organisms. This method is, in particular, applicable to photosensitive cells, i.e. capable of inducing a metabolic activity in response to a natural or artificial illumination, under mixotrophic conditions, in particular of protists. The method makes it possible to obtain both a high concentration of biomass and an enrichment of the cells thus cultured in molecules of interest, in particular in lipids and/or in carotenoids and, more particularly, in eicosapentaenoic acid (EPA) and/or in docosahexaenoic acid (DHA) and/or in arachidonic acid (ARA), and/or in a-linolenic acid (ALA) and/or in linoleic acid and/or oleic acid.

### Preamble

The production of raw materials by biological sources is becoming increasingly important given the current ecological context. Animal and plant cells, as well as microorganisms are now used as producers of molecules which are useful for the agri-food (for example, alcohols, organic acids, fatty acids, polysaccharides, pigments, amino acids, enzymes or vitamins), pharmaceutical (antibiotics, compounds with a pharmacological activity, exogenous or recombinant proteins), cosmetic (pigments, organic compounds such as acids, polysaccharides and fatty acids) and petrochemical industries (biofuels or intermediate products which can be converted to fuel, lubricants and bioplastics).

For example, protists are currently the subject of numerous industrial projects since certain species are capable of accumulating or secreting significant quantities of recoverable compounds, like lipids, in particular polyunsaturated fatty acids.

The production of the molecules of interest by cells is carried out on the industrial scale in general, by fermentation methods. In order to implement fermentation on an industrial scale, several factors must be taken into account, for example, for protists, the culture conditions (autotrophic, mixotrophic or heterotrophic) depending on the strain, the temperature, the lighting conditions and the size of the fermenters. For example, cultures may also be carried out in one-litre containers, in a laboratory, in photo-bioreactors, and in 100,000-litre containers or in open ponds (several hectares). However, the costs of energy and other resources such as manpower and the ease of monitoring the culture must be taken into account for developing ideal culture conditions.

In any case, it is desirable that the cells are cultured under optimum conditions for increasing the yield of the molecules to be produced.

Three types of fermentation methods exist which are commonly used in the field of biomass production, namely,

the discontinuous (or “batch”) mode:

The tank is filled with the sterilized culture medium, then the inoculum. The fermentation then takes place without the supplementary major addition of medium (mainly the pH correctives and anti-foaming agents). The volume remains constant and the productivity of biomass is relatively low. At the end of fermentation, the fermenter is emptied and its content is replaced.

the semi-continuous (or “fed-batch”) mode:

This production method is based on progressive feeding with nutritive substrate during the culture. This mode is generally used to achieve a high cell density in the fermenter.

the continuous mode:

In this method, a fermenter is used which is commonly called a “chemostat” into which a fresh culture medium is continuously added, while the culture liquid is continuously drawn-off in order to keep the culture volume constant.

The growth rate of the cells can be monitored by modifying the rate of feeding with nutritive matter while respecting the physiological limits of each type of cell or strain (depending on its specific maximum growth rate (μmax) under the specific process conditions).

The continuous mode allows the culture of the microorganisms in a given state of physiological equilibrium corresponding to an equilibrium between the growth of the biomass and the production of metabolite(s) of interest.

In the state of equilibrium, the cells grow at a constant rate and all the parameters of the culture remain constant (pH, volume, dissolved oxygen concentration, concentrations of nutritive elements and products, cell density, etc.).

The continuous mode has, in particular, the following advantages:

the overall production cost of biomass is reduced (less cleaning, sterilization and culture operations, immobilization of equipment at fermentation level);

the productivity (quantity of biomass produced per hour) is increased;

the continuous recovery of the biomass makes it possible to facilitate operations downstream by optimizing the dimensioning of the equipment.

However, the continuous methods also have drawbacks, in particular when the production of the molecule of interest is dissociated or partially dissociated from the cellular growth phase. Thus, when a substance of interest is produced outside this optimum growth phase (for example at the end of culture, during the stationary phase of the culture), it is more difficult to obtain performances that are compatible with economic interests.

Other constraints linked with the continuous mode are:

management of the effluents in greater quantity (lower concentration of biomass),

loss of unconsumed raw materials (residual substrates) can also have an impact on the methods downstream (extraction, drying etc).

U.S. Pat. No. 6,156,570 describes a method of cell culture in suspension, under heterotrophic conditions, in which the culture is initiated in semi-continuous mode and continued in continuous mode. This method makes it possible to modify the cellular energy metabolism, resulting in a low production of lactate, this which makes it possible to maintain a high cellular concentration. The cells used are preferably mammalian cells, or other animal sources (insects, fish etc.).

Application WO2003/020919 relates to a system with several units for the culture of animal or plant cells at high cell density, in suspension, with a view to obtaining biological products. This method uses in particular a pre-culture fermenter in semi-continuous mode and a culture fermenter in continuous mode. The method described is based on the spatial arrangement of the different units making it possible to implement them in synergic manner.

Application US2009/0263889 relates to a culture method under heterotrophic conditions for protists, in particular of the genera Schizochytrium or Thraustochytrium, for the production of cellular lipids such as DHA. The culture is initiated in discontinuous mode and continued in continuous mode, or comprises an intermediate step of culture in semi-continuous mode.

Application WO2012/074502 describes methods and systems for the culture of different photo-autotrophic microorganisms, in particular algae, diatoms, cyanobacteria, photobacteria or plants, in which a “biotic” (originating from another organism) or “abiotic” (inorganic or produced by other means) stimulation agent is used to generate or increase the production of metabolites. This stimulation agent can be used in combination with an illumination. This method combines the conditions of photo-autotrophic and mixotrophic, or photo-autotrophic and heterotrophic culture.

It has now been found that a method for the production of biomass comprising the culture of cells in continuous mode under mixotrophic or heterotrophic conditions in a fermenter (“growth” step), then continuous feeding and alternatively into fermenters operating in semi-continuous mode with the cells produced and their culture under mixotrophic or heterotrophic conditions in said fermenters (“maturation” step), makes it possible to obtain both an increase in the productivity of biomass and of molecules of interest.

Advantageously, such a production method has in particular the following advantages:

the biomass produced in continuous mode has a significant growth rate;

the cell mass is significantly increased by accumulation of molecules of interest during the culture in semi-continuous mode;

the phases of growth and production of molecules of interest are physically separated, which also allows an optimization of the culture parameters for each of the steps (light, temperature, pH, pO₂, stirring, etc.);

the growth phase in continuous mode then feeds maturation fermenters and makes it possible to considerably reduce the production shutdown phases associated with cleaning and sterilization which occur during discontinuous (“Batch”) cultures or semi-continuous (“Fed-Batch”) cultures;

in comparison to a continuous production mode, the method of the invention makes it possible to obtain an optimum composition of the biomass produced, i.e. the use of physico-chemical parameters during the maturation phase which are orientated for the accumulation of the molecule of interest makes it possible to considerably improve the productivity in comparison to production in continuous mode;

According to the method of the invention, there are less effluents in comparison to a continuous mode as the maturation phase makes it possible to increase the yield of biomass per litre and therefore to reduce the quantity of effluents to be retreated for an equivalent quantity of biomass.

The molecules of interest the production of which is sought are, in particular, pigments, such as fucoxanthin, astaxanthin, zeaxanthin, canthaxanthin, echinenone, beta-carotene and phoenicoxanthin, (exo-) polysaccharides, amino acids, vitamins, lipids, in particular fatty acids, and more particularly polyunsaturated fatty acids. Among these, certain highly unsaturated fatty acids (HUFA) from the omega-3 series (“polyunsaturated fatty acids” PUFA-w3), in particular eicosapentaenoic acid (EPA or C20:5 ω3) as well as its precursor, linolenic acid (ALA or C18:5 ω3.) and docosahexaenoic acid (DHA or C22:6ω3), and from the omega-6 series (PUFA-ω6), in particular arachidonic acid (ARA or AA or also eicosatetraenoic acid C20:4 ω6), as well as its precursor, linoleic acid (C18:21 ω6), have a recognized nutritional importance and have strong potential in terms of therapeutic applications. The mono-unsaturated fatty acids, in particular oleic acid, are also of interest.

Fish oils, from the fishing industry, are currently the main commercial source of this type of fatty acids. However, while these oils find new applications (food supplement in aquaculture, incorporation in margarines), marine halieutic resources are becoming scarce because of intensive fishing activity.

Therefore, new sources of these fatty acids such as EPA, DHA, ALA and ARA have to be sought in order to meet, in the future, the increasing market demand for this type of polyunsaturated fatty acids.

Short chain fatty acids (C1 to C7) for example acetic, propanoic and butyric acids, or medium chain fatty acids (C8 to C12), for example lauric acid, are also molecules originating from microorganisms and in particular from protists.

Moreover, the carotenoids, such as astaxanthin, zeaxanthin, canthaxanthin, echinenone, beta-carotene and phoenicoxanthin, lutein or fucoxanthin, are also molecules of interest produced by microorganisms. They are generally used as pigments, but they also have an important role in human health as antioxidants. Finally, they have the ability to stimulate the immune system.

Lutein, as well as zeaxanthin in much greater quantities, are the only carotenoids which are absorbed in the blood after ingestion and accumulate in the human retina. Lutein is associated with a possible reduction of the risks associated with ocular and cutaneous lesions caused by blue light. It is in particular associated with the prevention of age-related macular degeneration (ARMD).

Moreover, molecules known as “Mycosporine-like amino acids” or “MAAs”, are molecules of interest produced by microbes such as marine bacteria, cyanobacteria, fungi and various other marine organisms.

Moreover, the exo-polysaccharides produced by certain microorganisms are molecules of interest as they can enter into the composition of certain agri-food preparations as texturizers; or also as components of certain cosmetic formulations, for example as mattifying agents or texturizers.

The method of the invention relates to the culture of any type of cells, in particular the culture of protists for the production of fatty acids and/or of pigments on an industrial scale.

Most of the species of protists encountered in fresh water or in the oceans are generally autotrophic, i.e. they can only grow by photosynthesis. For the latter, the presence of organic carbon-containing substrates in their environment is not favourable to them and does not improve their growth. However, a certain number of species of protists, of very varied families and origins are found to be not strictly autotrophic. Thus, some of them, called heterotrophs, are capable of developing in the total absence of light, by an oxidative metabolism (fermentation or respiration), i.e. by using an organic carbon-containing substrate, such as sugars, as sole source of carbon and energy.

Other species of protists, for which photosynthesis remains indispensable to their development, are capable of using both photosynthesis and organic matter present in their environment. These intermediate species, called mixotrophs, can exploit the presence of light and organic carbon-containing substrates.

This particular feature of the so-called “mixotrophic” algae seems to be related to their metabolism, which allows them to carry out photosynthesis and oxidative metabolism simultaneously. Both types of metabolism coexist with an overall positive effect on the growth of the algae [Yang C. et al. (2000) Biochemical Engineering Journal 6:87-102].

Other organisms considered as mixotrophs exist. For example, Paracoccus pantotrophus bacteria, protists such as Euglena, and the plant Dionaea muscipula, are capable of drawing energy from light. The culture method according to the invention applies to the cells of organisms called “mixotrophs”.

Among the mixotrophic species, there can also be mentioned the case of the “photo-heterotrophic” species, which only develop by an oxidative metabolism (fermentation or respiration), but the light of which has an impact on the cell via photoreceptors, such as for example phototropism (ability to orientate with respect to light), the triggering of sexual reproduction cycles or more generally when the light generates a modification in the metabolism. These mechanisms are known in numerous organisms such as for example filamentous fungi of marine origin or also protists.

In the present invention, by “mixotrophic” cell, organism or microorganism, is meant cells, organisms or microorganisms capable of growing by using the light and an organic carbon-containing source, and also microorganisms and cells also called “photo-heterotrophic” which use light for a function other than growth, such as for example the production of molecules like carotenoids. Thus, the “mixotrophic” cells are capable of inducing a metabolic activity in response to natural or artificial illumination, under mixotrophic conditions.

In any event, it is desirable for the microorganisms, or cells, to be cultured under optimum conditions for increasing the yield of the molecules of interest, in particular, of fatty acid(s) and/or of pigments to be produced.

In particular, it would be desirable to be able to obtain yields of biomass, lipids and carotenoids greater than those described in the prior art for industrial exploitation which is more efficient and more cost-effective. Thus, it is preferable to have a yield which is as high as possible (for example above 80 g/l of dry matter, more than 30% of fatty acids by weight with respect to the total weight of dry matter and more than 0.1% by weight of carotenoids with respect to the total weight of dry matter).

### DETAILED DESCRIPTION

According to a first aspect, the invention therefore relates to a method for the production of biomass, comprising:
a) the culture of the cells in continuous mode under mixotrophic or heterotrophic conditions in a fermenter, called the “top fermenter”, then
b) the continuous feeding of n fermenters, called “maturation fermenters” operating in semi-continuous mode, n being an integer greater than 2, preferably from 2 to 10, with the cells produced in step a) and their culture under mixotrophic or heterotrophic conditions, preferably mixotrophic conditions.

According to an embodiment, the invention relates to a culture method which comprises a step of mixotrophic or heterotrophic growth and a step of accumulation of molecules of interest under mixotrophic conditions.

By “mixotrophic conditions” is meant a culture with a light supply and a supply of organic carbon-containing substrate.

By “heterotrophic conditions” is meant a supply of organic carbon-containing substrate and in darkness.

By “protists” is meant all the unicellular eucaryotic microorganisms. The microalgae (Chorella, Tetraselmis, Nitzschia etc.), the unicellular fungi (Schizochytrium, Aurantiochytrium etc.) or the heterotrophic flagellates (Crypthecodinium etc.) form part of the group of protists.

By “strain” is meant not only natural strains, but also the mutants of said natural strains.

By “mutants” is meant an organism derived from the original strain, the gene pool of which has been modified either by a natural method, or by physico-chemical methods known to one skilled in the art being able to generate random mutations (UV etc.,), or by genetic engineering methods.

Step a) is the growth phase of the cells making it possible to attain a significant population. In general, a specific culture medium (feed solution for the cells) is chosen to attain this goal, depending on the type of cells cultured, and the molecule(s) of interest to be produced. For example, in the case of the protists, the culture medium is enriched with a feed solution containing macronutrients, trace elements and vitamins. The medium contains an organic carbon-containing substrate, which allows culture in heterotrophic or mixotrophic mode.

Step b) is a phase of accumulation of molecules of interest. Preferably, this phase uses a second feed solution making it possible to preferentially orient the metabolism towards the accumulation of these molecules of interest. For example, in case of production of lipids by protists, this orientation can be done by increasing the ratio of the concentration of the carbon to that of the easily absorbed nitrogen (C/N ratio).

In practice, a first fermenter operating in continuous mode allows the growth (step a)) of the cells, and the outgoing flow (or draw-off) from this growth fermenter supplies the maturation tanks where the biomass is enriched with molecules of interest (step b)).

The method can be described as a method called “uncoupled” as the growth phases according to step a) and maturation according to step b) are physically and temporally separated.

Step a) of culture of the cells in continuous mode of the method for the production of biomass according to the invention, or “growth step”, can be implemented in a fermenter known to one skilled in the art, such as for example, the “chemostat”.

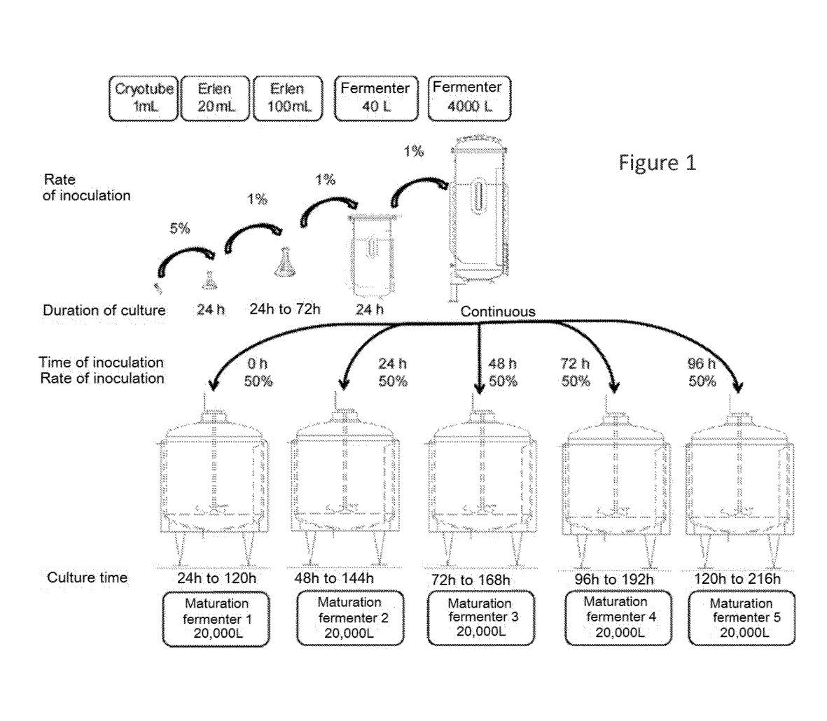

The start of the culture of step a) can be carried out in a standard fashion (see FIG. 1). Pre-culture steps can be carried out upstream, so as to obtain the desired cell concentration for starting the continuous production of biomass, namely between 2 and 50 g/L, preferably between 5 and 40 g/L.

In general, the increase in scale from the cryotube is carried out in several pre-culture steps. For example, it can be carried out in three steps, two steps in an Erlenmeyer (preculture 1 and preculture 2), and one step in a fermenter (pre-fermenter). The growth in the Erlenmeyer takes place in discontinuous (“batch”) mode. In general, the growth in the pre-fermenter also takes place in discontinuous mode.

For example, the culture can pass progressively from a culture in the 20 mL Erlenmeyer to a culture in the 2 L Erlenmeyer, then to a culture in the fermenter (pre-fermenter) in discontinuous (“batch”) mode, then to a culture in a fermenter in continuous (“top fermenter”) mode. For example, the fermenter in discontinuous mode has a volume of 40 L and the top fermenter has a volume of 4,000 L.

The start of the culture in the top fermenter has an establishment phase during which the continuous regime is not attained, i.e. the growth of the cells is not stabilized, or is not in phase with the rate of feed with fresh medium. It is a phase of “attaining equilibrium” that can also be called “phase of attaining a steady state”. This “phase of attaining a steady state” has a duration which depends on the growth rate of the microorganisms cultured or more precisely on the specific growth rate or μmax. By way of example, this duration can be comprised between 5 and 200 hours, preferably between 10 and 100 hours.

Generally, once the steady state is attained, a draw-off rate from the top fermenter to the maturation fermenter(s) is established at a flow rate equivalent to the rate of feeding with fresh culture medium. This draw-off feeds a first maturation fermenter until the desired volume is transferred, then the draw-off is redirected to another maturation tank. One skilled in the art will know how to determine the volume of inoculum necessary according to the type of cells to be cultured, the molecule of interest to be produced and the capacities of the maturation tanks according to an embodiment. This volume of inoculum can be comprised between 10 and 90% of the capacity of the maturation fermenter, depending on the cells cultured; for example approximately 20%, 30%, 40%, 50%, 60%, 70%, or 80% of the capacity of the maturation fermenter.

The draw-off to the maturation fermenters is carried out in a standard fashion using known equipment.

Generally, a culture medium different from that used during step a) is employed during the maturation phase. According to the different embodiments, this culture medium is supplied in the maturation fermenter simultaneously to, or prior to, or subsequently to the inoculum originating from the draw-off from the top fermenter.

The volume of the culture in the fermenter of step a) is determined as a function of the final volume of culture desired for the cultures in the maturation fermenters. In general, the top fermenter has a volume of 5 to 100%, preferably 10% to 50% and more preferentially approximately 20% of the volume of the maturation fermenter that it feeds. For example, a top fermenter having a volume of 4,000 L can feed a maturation fermenter of 20,000 L. If the n maturation fermenters have different volumes, it is the volume of the largest fermenter which will determine the volume of the top fermenter.

The culture medium used in step a) is chosen according to the characteristics of the cells cultured.

For the culture of bacteria, fungi or yeasts, a specific medium can be developed for a defined method. However, a minimum medium or an enriched nutritive medium can be used and there can be mentioned by way of example Verduyn culture medium for the growth of yeasts or protists, or LB (Lysogeny Broth) medium for the growth of bacteria. For the culture of plant cells, for example, a medium of Murashige and Skoog type can be used. For the culture of animal cells, DMEM (Dulbecco's Modified Eagle Medium) culture medium can be mentioned by way of example.

For the culture of microorganisms in general, of bacteria, of protists or of fungi in particular, the culture medium for step a) in general contains a source of nitrogen and of phosphate, as well as an organic carbon-containing substrate. The latter allows culture in mixotrophic mode.

An example of this type of medium is modified Verduyn medium (sea salts 15 g/L, (NH₄)₂SO₄ 3 g/L, KH₂PO₄ 1 g/L, MgSO₄.7H₂0, 0.5 g/L, Na₂EDTA 24 mg/L, ZnSO₄.7H₂O 3 mg/L, MnCl₂.2H₂O 3 mg/L, Na₂MoO₄.2H₂O 0.04 mg/L, FeSO₄.7H₂O 10 mg/L, pantothenate 3.2 mg/L, thiamine hydrochloride 9.5 mg/L, vitamin B12 0.15 mg/L, anti-foaming agent 0.1mL/L) to which an organic carbon-containing substrate is added.

Another example of a culture medium is: sea salts 25 g/L, KNO₃ 3 g/L, NaH₂PO₄ 1 g/L, MgSO₄.7H₂0, 2.5 g/L, Na₂EDTA 24 mg/L, Na₂SiO₃ 9H₂O 0.3 g/L, ZnSO₄.7H2O 1 mg/L, MnCl₂.2H₂O 1 mg/L, Na₂MoO₄.2H₂O 0.04 mg/L, FeSO₄.7H₂O 10 mg/L, yeast extracts 2 g/L, Biotin 0.15 mg/L, thiamine hydrochloride 9.5 mg/L, Vitamin B12 0.15 mg/L, anti-foaming agent 0.1 mL/L) to which an organic carbon-containing substrate is added.

According to an embodiment of the invention, the organic carbon-containing substrate has a concentration from 5 mM to 1.1 M (from 0.9 to 200 g/L), preferably from 50 mM to 800 mM (from 9 to 144 g/L).

This organic carbon-containing substrate preferentially comprises, in the pure form or in a mixture: glucose, cellulose derivatives, lactate, starch, lactose, saccharose, acetate and/or glycerol.

The organic carbon-containing substrate contained in the culture medium may consist of complex molecules or a mixture of substrates. The products resulting from the biotransformation of starch, for example starting from maize, wheat or potato, notably starch hydrolysates, which consist of small sized molecules, for example, form organic carbon-containing substrates suitable for mixotrophic culture of the cells according to the invention.

Additional substrate is added to the culture medium during the culture method in order to maintain a sufficient concentration for the culture. One skilled in the art knows how to ensure that the level of organic carbon-containing substrate is maintained.

Preferably, the organic carbon-containing substrate comprises glucose, glycerol, at a concentration equivalent to or greater than 300 mM.

By way of example, the culture according to step a) of the strains of protists of the genus Schizochytrium or Aurantiochytrium for the production of DHA can be carried out in a saline culture medium (sea salt 10-20 g/L, preferably 15 g/L) with a glucose concentration from 5 to-200 g/L, preferably 30 to 100 g/L and more preferentially from 30 to 60 g/L. According to an embodiment, the culture medium also contains macronutrients, such as magnesium salts and potassium salts, at a concentration from 1 to 50 g/L, preferably 2 to 25 g/L. According to an embodiment, the medium contains ammonium salts, preferably at a concentration of 1.5 to 7.5 g/L. It also contains trace elements used in a standard fashion for the culture of protists, such as the salts of manganese, zinc, cobalt, molybdenum and copper, nickel and iron. Typical values for the concentration of these trace elements are from 0.02 to 15 mg/L for each trace element. For example, the magnesium and zinc salts can be present at 2-4 mg/L, the cobalt and molybdenum salts can be present in a small quantity, such as 0.01-0.04 mg/L, preferably between 0.02 and 0.03 mg/L. The copper and nickel salts can be present at 1-4 mg/L, preferably at 1-2 mg/L. In general, the iron salts are present at a more significant level than the other salts, for example at a level of 8-15 mg/L.

According to another embodiment, the magnesium salts are present at 70-300 mg/L. According to an embodiment, the medium also comprises vitamins such as thiamine, vitamin B12, panthotenate and optionally a stabilizing agent.

According to an embodiment of the invention which is suitable for attaining very high densities of Aurantiochytrium (1 to 3×10⁹ cells/ml), the culture medium called “initial medium” in Table 1 below can be used.

TABLE 1
Example of culture media useful for the culture of the strains of
Aurantiochytrium, according to step a) of the culture method.

| | Initial medium | Feed solution |
|---|---|---|
| Carbon source | 1 to 1.6 CMol | 3.7 CMol |
| NH4⁺ | 1.5 to 7.5 g/L | 5 to 10 g/L |
| Sea salt | 5 to 30 g/L | 10 to 30 g/L |
| Ca | 25 to 70 mg/L | 100 to 200 mg/L |
| Mn | 1 to 10 mg/L | 4 to 20 mg/L |
| Zn | 0.1 to 1 mg/L | 0.5 to 2 mg/L |
| S | 0.5 to 1.5 g/L | 2 to 4 g/L |
| Co | 0.1 to 1 mg/L | 0.5 to 2 mg/L |
| Mo | 0.01 to 0.5 mg/L | 0.05 to 1.5 mg/L |
| Cu | 0.05 to 0.3 mg/L | 0.25 to 0.8 mg/L |
| Ni | 0.02 to 0.2 mg/L | 0.1 to 0.5 mg/L |
| K | 0.5 to 2 g/L | 3 to 5 g/L |
| Mg | 70 to 300 mg/L | 250 to 750 mg/L |
| P | 0.3 to 1 g/L | 1.3 to 2.5 g/L |
| Fe | 5 to 10 mg/L | 15 to 25 mg/L |
| Thiamine | 15 to 50 mg/L | 50 to 150 mg/L |
| Vitamin B12 | 0.2 to 0.7 mg/L | 0.7 to 2 mg/L |
| Panthotenate | 0.05 to 0.15 mg/L | 15 to 45 mg/L |
| Stabilizing agent | To be decided by the | To be decided by the |
| (EDTA type) | laboratory assistant | laboratory assistant |

The “initial medium” corresponds to the medium which can be used for starting the continuous culture. Then “the feed solution” can be used for continuously supplying the fermenter.

Generally, when the biomass concentration in the top fermenter has attained equilibrium, for example for protists, bacteria and yeasts, between approximately 5 and 100 g/L, preferably between approximately 10 and 70 g/L of biomass concentration, the draw-off from the top fermenter serves to continuously and successively feed the different maturation fermenters operating in semi-continuous mode. The culture of the cells is thus maintained under mixotrophic or heterotrophic conditions in said fermenters (“maturation” step), with a view to the production of molecules of interest. Thus, generally, feeding the maturation fermenters according to step b) starts when the top fermenter is in a steady state with a stable cell density. For example, for the culture of protists, this cell density could be between 10⁷ and 10⁹ cells per mL. For yeasts, this cell density could be between 10⁸ and 10⁹ cells per mL. For bacteria, this cell density could be between 10⁹ and 10¹⁰ cells per mL.

Feeding the maturation fermenters according to step b) is (in general) carried out successively, i.e. a maturation fermenter n is fed with the draw-off from the top fermenter before passing onto the following maturation fermenter, n+1.

The volume of inoculum used for inoculating the maturation fermenter is in general dependant on the cell cultured and on the molecule(s) of interest to be produced and on the volume necessary in the maturation fermenter before moving on to the step of production of the molecule of interest. For example, in the case of protists, yeasts and bacteria, the volume can represent between 10 and 70% of the final volume of the cell suspension in the maturation fermenter, preferentially between 20 and 60%, more preferentially between 30 and 50%.

Generally, once the desired volume of inoculum has been attained in the maturation fermenter n, the second (n+1) maturation fermenter (or “maturing vessel”) is fed, then the third (n+2) maturation fermenter is fed, etc.

According to an embodiment, the culture in the maturation fermenter is stopped, then the fermenter is emptied when the production of the molecule(s) of interest has attained the desired level, for example, in the case of protists of the genus Aurantiochytrium, when the DHA titre is between 5 and 30 g/L and/or the carotenoid pigment titre between 0.1 and 10 mg/g of dry biomass.

Emptying is carried out in a standard fashion to devices allowing treatment of the biomass with a view to the extraction of the molecule(s) of interest. After having been emptied, the maturation fermenter is cleaned, generally with a specific piece of equipment which is incorporated into the tanks of the fermenters, then it is sterilized.

For example (see Table 2 and the diagram according to FIG. 1), with a culture time in the maturation fermenters of 72 h, on the first day, D, the maturation fermenter 1 is filled and the other maturation fermenters are waiting; on D+1, the maturation fermenter 2 is filled (culture N+2) and the maturation fermenter 1 is in the process of culturing; on D+2, the maturation fermenter 3 is filled and the maturation fermenters 1 and 2 are in the process of culturing; on D+3, the maturation fermenter 1 is cleaned, the maturation fermenter 4 is filled and the maturation fermenters 2 and 3 are in the process of culturing.

TABLE 2

| | Maturation | Maturation | Maturation | Maturation |
|---|---|---|---|---|
| | fermenter 1 | fermenter 2 | fermenter 3 | fermenter 4 |
| Day D | Culture N | Waiting | Waiting | Waiting |
| Day | Culture N | Culture N + 1 | Waiting | Waiting |
| D + 1 | | | | |
| Day | Culture N | Culture N + 1 | Culture N + 2 | Waiting |
| D + 2 | | | | |
| Day | Cleaning and | Culture N + 1 | Culture N + 2 | Culture N + 3 |
| D + 3 | sterilization | | | |
| Day | Culture N + 4 | Cleaning and | Culture N + 2 | Culture N + 3 |
| D + 4 | | sterilization | | |
| Day | Culture N + 4 | Culture N + 5 | Cleaning and | Culture N + 3 |
| D + 5 | | | sterilization | |

The number of maturation fermenters fed by the top fermenter is greater than 2, and, generally, this number is limited only by the space available for storing said fermenters and their associated equipment. In general, the number of maturation fermenters is from 2 to 10, preferably from 2 to 6.

According to an embodiment of the invention, the maturation culture time varies between 10 and 200 hours, preferably between 20 and 100 hours. The cycle illustrated in the table varies as a function of this time. For example, for bacterial cells, the maturation culture time can vary between 15 and 72 hours. For example, for plant cells, the maturation culture time can vary between 48 and 200 hours.

For the culture of protists, bacteria and yeasts, this culture step in semi-continuous mode is generally carried out for 24 to 200 h in each fermenter, preferably for 48 h to 96 h, more preferentially 72 h to 96 h.

The composition of the culture medium for step b) allows a residual growth to be maintained, while promoting the accumulation of the molecules of interest. By “residual growth” is meant a multiplication of the cells at a growth rate comprised between 1 and 90% of the growth rate of step a) but which allows an improvement in productivity in terms of biomass and of the molecule(s) of interest.

For example, for the production of lipids by protists, the growth rate in continuous culture is from 0.05 to 0.20 h⁻¹, preferably 0.10 h⁻¹.

The growth rate characterizes the increase in the population over time. The growth rate (μ) is calculated according to the formula:

μ=(ln N2−ln N1)/(t2−t1)

where N1=quantity of biomass at time t1, N2: quantity of biomass at time t2. The quantity of biomass can be expressed in optical density, cell density or dry mass. The times t1 and t2 are expressed in hours. The value of μ is expressed in h⁻¹ (1/hour).

The culture medium used for the fermentation according to step b) of semi-continuous culture is chosen according to the nature of the cells cultured. Preferably, this culture medium makes it possible to preferentially direct the metabolism of the cultured cells towards the accumulation of one or more molecule(s) of interest. For certain cells, the limitation of an element in the culture medium is a method which makes it possible to promote the accumulation of a molecule. By way of example, the protists which can increase the accumulation of the lipids during nitrogen limitation can be mentioned. By nitrogen limitation is meant a concentration of nitrogen in the culture medium insufficient to ensure the normal growth of the cells, and which results in a modification of the metabolism which promotes the accumulation of carbon in the form of lipids. This modification of the metabolism can also be obtained by increasing the C/N ratio (ratio of the concentration of carbon to that of easily absorbed nitrogen).

However, the culture medium contains an organic carbon source, which allows mixotrophic or heterotrophic growth. Typically, for a protist culture, the organic carbon is present at a concentration from 10 to 90 g/l (from 55 mM to 500 mM).

In general, the culture according to step b) of maturation of the protists for the production of lipids or pigments can be carried out in a saline culture medium (sea salt 10-30 g/L) comprising approximately 20 to 200 g/L (0.1 to 1.1 M) of organic carbon-containing substrate, approximately 1 to 15 g/L of ammonium and approximately 0.9 to 3.5 g/L of phosphate. By way of example for the production of DHA by the strain Aurantiochytrium, the culture medium (reconstituted from the volume of the cell suspension originating from the top fermenter and from the volume of concentrated feed solution) comprises from 100 to 200 g/L of glucose, approximately 5 to 10 g/L of ammonium and 1 to 2 g/L of phosphate. According to an embodiment of the invention, the culture medium also contains macronutrients, trace elements and vitamins.

According an embodiment, the nutritive solution used in the maturation step contains a carbon source (for example glucose or glycerol), a nitrogen source (for example (NH₄)₂SO₄, a phosphate source (for example KH₂PO₄). Preferably, the ratio between the C (carbon), the N (the nitrogen) and the P (the phosphate) (C/N/P) is 530:11:1 always with the possibility of having up to times as much nitrogen (N) more or less, and/or up to twice as much phosphate (P) more or less, for example the following ratios can be used 530:22:1.530:5.5:1; 530:22:0.5; 530:11:2; 530:22:2; 530:5.5:0.5; 530:11:0.5; 530:5.5:2.

In general, the culture according to step b) of maturation of the strains of yeasts for the production of carotenoid pigments can be carried out in a culture medium comprising approximately 10 to 100 g/L of organic carbon-containing substrate, approximately 0.15 to 15 g/L of ammonium and approximately 0.9 to 9 g/L of phosphate. By way of example, for the production of astaxanthin by Rhodotorula glutinis, the culture medium comprises 20 g/L glucose, approximately 3 g/L of ammonium and 1 g/L of phosphate.

In general, the culture according to step b) of maturation of the strains of bacteria for the production of amino acids can be carried out in a culture medium comprising approximately 20 to 80 g/L of organic carbon-containing substrate, approximately 10 to 20 g/L of urea and approximately 1 to 10 g/L of phosphate. By way of example for the production of L-methionine by Brevibacterium flavum, the culture medium comprises 50 g/L glucose, approximately 10 g/L of urea and 5 g/L of phosphate.

According to an embodiment, step b) is carried out at a lower temperature than step a). The inventors have noted that, according to this embodiment, the quantity of lipids, in particular of DHA, produced is increased relative to a culture where step b) is carried out at the same temperature as step a). In fact, according to the method of invention, the optimum temperature for growth is greater than the optimum temperature for the production of DHA. Generally, step b) is carried out at 1 to 8 degrees less than the temperature at which step a) is carried out. For example, step a) of the culture can be carried out at 25° C., while step b) can be carried out at 18° C., 19° C., 20° C., 21° C., 22° C. or 23° C. For example, step a) of the culture can be carried out at 22° C., while step b) can be carried out at 18° C., 19° C., 20° C., 21° C. For example, step a) of the culture can be carried out at 27° C., while step b) can be carried out at 20° C., 21° C., 22° C., 23° C., 24° C., 25° C. or 26° C.

A low temperature also promotes the accumulation of pigments.

The culture method according to the invention is carried out under mixotrophic conditions and/or under heterotrophic conditions, i.e. step a) and step b) can be carried out independently under heterotrophic conditions or under mixotrophic conditions. According to an embodiment, step a) is carried out under heterotrophic conditions and step b) under mixotrophic conditions. According to another embodiment, step a) is carried out under mixotrophic conditions and step b) under heterotrophic conditions. According to another embodiment, steps a) and b) are carried out under heterotrophic conditions. For example, this embodiment is suitable for protists, bacteria or yeasts which grow well under heterotrophic conditions. According to another embodiment, steps a) and b) are carried out under mixotrophic conditions. For example, this last embodiment is suitable for protists or bacteria, yeasts and other photosensitive cells which are therefore capable of drawing energy from light. As an example, the protists of the genus Schizochytrium or Aurantiochytrium, cyanobacteria of the genus Nostoc or yeasts of the genus Rhodotorula can be mentioned.

More particularly, the mixotrophic conditions can be conditions of illumination that is discontinuous and/or variable over time. By “discontinuous illumination” is meant illumination punctuated by periods of darkness. The illumination can in particular be in the form of flashes. A flash, within the meaning of the invention, is an illumination with light of short duration, i.e. of less than 30 minutes. The duration may be less than 15 minutes, preferably less than 5 minutes or even more preferentially less than 1 minute. According to certain embodiments of the invention, the duration of the flash may be less than a second. For example, the duration of the flash may be 1/10 of a second, or 2/10 of a second, or 3/10 of a second, or 4/10 of a second, or 5/10 of a second, or 6/10 of a second, or 7/10 of a second, or 8/10 of a second, or 9/10 of a second. The illumination with light, or the flash, generally lasts longer than 15 seconds. The duration of the flash is generally comprised between 5 seconds and 10 minutes, preferably between 10 seconds and 2 minutes, more preferentially between 20 seconds and 1 minute. These flash durations are suitable for light supplies of “low frequency”;

According to this last embodiment, where the illumination regime (the supply of light) is of “low frequency”, the number of flashes is comprised between about 2 and 3,600 per hour. It may be, for example, comprised between 100 and 3,600 flashes per hour. It may also be comprised between 120 and 3,000, or between 400 and 2,500, or between 600 and 2,000, or between 800 and 1,500 flashes per hour. It may also be comprised between 2 and 200, preferentially between 10 and 150, more preferentially between 15 and 100, and even more preferentially between 20 and 50 per hour.

According to an embodiment, a flash has a duration between 1/150,000 second and 1/1000 second. These flash durations are appropriate for “high frequency” illumination regime; i.e. with flash frequencies from 150 kHz to 1 kHz respectively.

According to this embodiment where the illumination conditions (the supply of light) are of “high frequency”, the flashes can occur between 3.6×10⁵ and 5.4×10⁹ times per hour. In this case, the light variation has a frequency of between 1 kHz and 150 kHz, i.e. between 1,000 and 150,000 flashes per second. The supply of light according to this last embodiment of the invention is called “high frequency”.

The number of flashes per hour is selected as a function of the intensity and duration of the flashes (see below). In general, the intensity of the light supplied in the form of flashes is between 5 and 1,000 μmol·m⁻²·s⁻¹, preferably between 5 and 500 μmol·m⁻²·s⁻¹, or 50 and 400 μmol·m⁻²·s⁻¹, and more preferentially between 150 and 300 μmol·m⁻²·s⁻¹. 1 μmol·m⁻²·s⁻¹ corresponds to 1 μE m⁻²·s⁻¹ (Einstein), a unit often used in the literature.

According to another embodiment of the invention, the illumination may be variable, which means that the illumination is not interrupted by phases of darkness, but instead the light intensity varies over time. This variation of the light intensity is regular and may be periodic or cyclic. According to the invention, light may also be supplied combining phases of continuous and discontinuous illumination.

By “variable illumination” is meant that the intensity of the light varies regularly at least twice per hour. An example of the illumination conditions suitable for the method of the invention is described in French patent application No. 1057380.

The illumination preferably has variations in intensity, the amplitude of which is generally comprised between 5 μmol·m⁻²·s⁻¹ and 2,000 μmol·m⁻²·s⁻¹, preferably between 50 and 1,500, more preferentially between 50 and 200 μmol·m⁻²·s⁻¹.

According to a preferred embodiment, the illumination has variations in intensity the amplitude of which is comprised between 5 and 1,000 μmol·m-2.s-1, preferably between 5 and 400 μmol·m-2.s-1, these variations taking place between 2 and 3600, preferably between 2 and 200 times per hour.

These variations can generally take place between 2 and 3,600 times per hour, preferably between 50 and 500 times, more preferentially between 100 and 200 times per hour. These culture conditions make it possible to supply a defined quantity of light. This light supply can comprise phases of discontinuous and/or variable illumination, with variations in intensity which can have identical or different amplitudes.

It appeared that variable and/or discontinuous illumination of the cultures, in particular when used in a culture in mixotrophic mode, had a favourable impact on the development of the cells or the production of molecules of interest, in particular of protists, procaryotic microorganisms, yeasts and plant or animal cells and made it possible to increase the productivity of the latter, notably as far as their production of molecules of interest, such as lipids and pigments is concerned. Without being bound by theory, the inventor believes that a discontinuous and/or variable light supply to the cells has the effect of causing a “stress” favourable to the growth and to the synthesis of lipids. This phenomenon may be partly explained by the fact that, in nature, cells tend to accumulate lipid reserves to withstand the constraints of their environment.

According to an embodiment of the invention, in the case of the mixotrophic conditions, the culture according to step a) is carried out in the presence of flashes. The intensity of the flashes is comprised between 50 and 200 μmol·m⁻²·s⁻¹, the duration of the flashes is comprised between approximately 1/10th of a second and five minutes, preferably between approximately a second and a minute and the number of flashes per hour can vary between 2 and 3,600, preferably between 2 and 360 per hour.

According to an embodiment of the invention, the culture according to step b) is carried out in the presence of flashes. The intensity of the flashes is comprised between 200 and 2,000 μmol·m⁻²·s⁻¹, the duration of the flashes is comprised between approximately 1/10th of a second and ten minutes, preferably between approximately 1/10th of a second and five minutes and the number of flashes per hour can vary between 2 and 3,600, preferably between 20 and 1000 per hour.

According to an embodiment of the invention, regardless of the illumination conditions, the intensity of the light supplied to the culture varies as a function of the cell density. The more dense the culture becomes, the more intense the light may be. The cell density is the number of cells per ml and it is measured by the techniques known to one skilled in the art.

For example, for protists, bacteria and yeasts, at the initial stage of the culture, when the cell density is between about 10⁷ and 5×10⁷ cells per ml and for a duration of flashes varying between 1 second and 60 minutes with 2 and 360 flashes per hour, the light intensity may be between 50 and 200 μmol·m⁻²·s⁻¹, preferably between 50 and 150 μmol·m⁻²·s⁻¹. When the culture attains a density between 10⁸ and 10⁹ cells per ml, the light intensity may be increased to between 100 and 500 μmol·m⁻²·s⁻¹, for example, preferably between 200 and 400 μmol·m⁻²·s⁻¹. When the culture, at the final stage, attains a density between 10⁹ and 10¹⁰ cells per ml, the light intensity may be increased to between 200 and 800 μmol·m⁻²·s⁻¹, for example, preferably between 200 and 500 μmol·m⁻²·s⁻¹.

For the culture of protists, bacteria and yeasts according to step b) for a duration of flashes varying between 1/10th of a second and 30 minutes with 2 and 3,600 flashes per hour, the light intensity can be increased up to between 200 and 2,000 μmol·m⁻²·s⁻¹, for example, preferably, between 300 and 1,000 μmol·m⁻²·s⁻¹.

According to certain embodiments, for example, when the duration of the flashes is for example less than a minute, or less than a second, the intensity of the light can be greater than the values mentioned above.

As detailed below (see Tables 3A and 3B), the genera of protists concerned by the method of the invention fall into two categories: Group 1 (“non-photo-synthetic”) and Group 2 “photo-synthetic”. Generally, in theory, the strains of Group 1 will require less, or can exploit light differently in order to grow and in order to produce the molecules of interest with respect to Group 2. On the other hand, the individual strains can have different light requirements. One skilled in the art knows how to adjust the exact parameters of light according to the strain in culture depending on its requirements.

According to an embodiment of the invention, the culture of the strains belonging to the genera Schizochytrium, Aurantiochytrium and Crypthecodinium (Group 1) is carried out with an intensity of light between 25 and 1200 μmol·m⁻²·s⁻¹, preferentially between 75 and 800 μmol·m⁻²·s⁻¹, more preferentially between 150 and 600 μmol·m⁻²·s⁻¹. According to this embodiment, the intensity of the light varies between 2 and 200 times per hour. The amplitude of the variations in intensity is comprised between 5 and 400 μmol·m⁻²·s⁻¹, 70 and 300 μmol·m⁻²·s⁻¹, and preferably between 100 and 200 μmol·m⁻²·s⁻¹.

According to an embodiment of the invention, the culture of the strains belonging to the genera Schizochytrium, Aurantiochytrium and Crypthecodinium (Group 1) is carried out with flashes.

For example, according to an embodiment, the culture of the genera of Group 1 is illuminated with flashes of an intensity of 200 μmol·m⁻²·s⁻¹, with 30 flashes per hour.

According to another embodiment of the invention, the culture of the strains belonging to the genera Tetraselmis, Scenedesmus, Chlorella, Nitzschia and Haematococcus (Group 2) is carried out with an intensity of light between 10 and 800 μmol·m⁻²·s⁻¹, preferentially from 25 to 600 μmol·m⁻²·s⁻¹ more preferentially from 75 to 250 μmol·m⁻²·s⁻¹. According to this embodiment, the intensity of the light varies between 2 and 3,600 times per hour. The amplitude of the variations in intensity is comprised between 5 and 400 μmol·m⁻²·s⁻¹, 70 and 300 μmol·m⁻²·s⁻¹, and preferably, between 100 and 200 μmol·m⁻²·s⁻¹. According to an embodiment of the invention, the culture of the strains belonging to the genera Tetraselmis, Scenedesmus, Chlorella, Nitzschia and Haematococcus (Group 2) is carried out with flashes.

For example, according to an embodiment, the culture of the genera of Tetraselmis and Scenedesmus is illuminated with flashes of an intensity of 100 μmol·m⁻²·s⁻¹, with 120 flashes per hour.

For example, according to an embodiment, the culture of strains of the genus Chlorella is illuminated with variable light, the intensity of the light varying between 50 and 100 μmol·m⁻²·s⁻¹, 30 times per hour.

For example, according to an embodiment, the culture of strains of the genus Haematococcus is illuminated with variable light, the intensity of light varying between 100 and 150 μE, 60 times per hour.

For example, according to an embodiment, the culture of strains of the genus Nitzschia is illuminated with flashes, of an intensity of 200 μE, 30 times per hour.

As described for the light intensity above, and according to an embodiment of the invention, the quantity of light supplied to the culture per hour may vary as a function of the cell density. At the initial stage of the culture when the cell density is between 10⁵ and 5×10⁵ cells per ml, the total light supply per hour is generally comprised between about 1,500 and 8,000, preferably 1,500 and 6,000 μmol·m⁻², more preferably between 2,000 and 5,000 μmol·m⁻². When the culture attains a density between 10⁶ and 10⁷ cells per ml, the total light supply per hour may be increased to between 6,000 and 67,000 μmol·m⁻², preferably between 6,000 and 50,000 and more preferably between 12,000 and 45,000 μmol·m⁻², for example. At the final stage of the culture, at a cell density between 10⁷ and 10⁸ cells per ml, the total light supply per hour may be increased to between 45,000 and 300,000, for example, preferably between 45,000 and 200,000 μmol·m⁻², and for example, yet more preferably, between 50,000 and 150,000 μmol·m⁻².

According to an embodiment of the invention, at the initial stage of the culture (at a cell density between 10⁵ and 5×10⁵ cells per ml), the culture is illuminated with 30 flashes per hour, each flash having a duration of 30 seconds and an intensity between 5 and 10 μmol·m⁻²·s⁻¹, which gives a total light supply per hour from 2,250 μmol·m⁻² to 4,500 μmol·m⁻². Then, at the intermediate stage (at a cell density between 10⁶ and 10⁷ cells per ml), the culture is illuminated with 30 flashes per hour, each flash having a duration of 30 seconds and an intensity between 15 and 50 μmol·m⁻²·s⁻¹, which gives a total light supply per hour of 13,500 to 45,000 μmol·m⁻². Then, at the final stage of the culture (at a cell density between 10⁷ and 10⁸ cells per ml), the culture is illuminated with 30 flashes per hour, each flash having a duration of 30 seconds and an intensity between 50 and 150 μmol·m⁻²·s⁻¹, which gives a total light supply per hour of 45,000 to 135,000 μmol·m⁻².

According to an embodiment of the invention, for example when the duration of the flashes is for example less than a minute, or less than a second, in the initial stage of culture (at a cell density between 10⁵ and 5×10⁵ cells per ml), the culture is illuminated with 30 flashes per hour, each flash having a duration of 10 seconds and an intensity between 50 and 100 μmol·m⁻²·s⁻¹, which gives a total supply of light per hour from 15,000 μmol·m⁻² to 30,000 μmol·m⁻². Then at the intermediate stage (at a cell density between 10⁶ and 10⁷ cells per ml), the culture is illuminated with 50 flashes per hour, each flash having a duration of 10 seconds and an intensity between 200 and 300 μmol·m⁻²·s⁻¹, which gives a total supply of light per hour of 100,000 to 150,000 μmol·m⁻². Then, at the final stage of the culture (at a cell density between 10⁷ and 10⁸ cells per ml), the culture is illuminated with 120 flashes per hour, each flash having a duration of 10 seconds and an intensity between 350 and 450 μmol·m⁻²·s⁻¹, which gives a total supply of light per hour of 420,000 to 540,000 μmol·m⁻².

The light supply to the cultures may be obtained by light sources distributed around the external wall of the fermenters. A clock triggers these lamps for defined illumination times. According to a preferred embodiment, the lighting of the cultures of step a) and/or b) is ensured by an illumination device internal to the fermenter. Thus, the effectiveness of the illumination is better relative to a configuration where the light penetrates via windows from sources arranged on the outside. The illumination devices can be arranged on the revolving assembly equipped with blades or on the tubular parts immersed in the mass to be treated, or also on the bottom or the top of the tank. According to a preferred embodiment of the invention, the illumination device is situated on the baffles inside the fermenter. Such a device is described in the French patent application No.1353641. The fermenter is thus equipped with a plurality of illumination sources borne by baffles, the latter having the function of preventing the formation of a vortex within the biomass under the action of the revolving mixing assembly. These illumination sources are preferably encapsulated, partially or completely in at least one part of these baffles, in a material that is compatible with the biomass and of a thickness that makes it possible to diffuse said light inside the tank.

A particularly suitable material is polysulphone which combines a good compatibility with food standards (including the American standards of the Food and Drug Administration, or abbreviated to FDA), a good heat transfer coefficient (allowing removal of the heat to the mass of micro-organisms and a semi-transparent character allowing good transmission of light, if the material has a thickness chosen between 1 mm and 5 cm; moreover, this material retains its properties after an optional heat treatment for sterilization or for cleaning with detergents or acid. If the combination of desired characteristics is modified, other materials can be chosen, for example polyurethane, polypropylene, an acrylic material or a polycarbonate.

Preferably, the illumination sources are light emitting diodes (abbreviated to

LED); they are light sources that are easy to control, both in their implementation and in their applications. Such sources can have very diverse emission spectra, as there are white LEDs (simulating sunlight), but also LEDs with a reduced spectral range (for example centred on red, blue or green light). Such illumination sources generate less heat than bulbs or lamps; moreover they have dimensions which are sufficiently small to be able to be located on the faces of the baffles without leading to an increased thickness which interferes with the main function of these plates.

The spectrum of the illumination sources is advantageously in the visible range, but can also, depending on requirements, be outside this visible range, for example in the UV (for example for applications involving sterilization), or in the infra-red (for example, in applications aimed at generating heat within the mass to be treated).

The illumination sources can have very varied control conditions (also termed monitoring or management conditions).

The method according to the invention has the advantage of increasing the yield of biomass obtained from the culture. This method also has the advantage of increasing the accumulation of molecules of interest. The following can be mentioned as examples of these molecules of interest: alcohols, including primary alcohols, secondary alcohols, esters of alcohols and fatty alcohols, fatty acids (FA) including short chain fatty acids (SOFA), long chain fatty acids (LCFA), very long chain fatty acids (VLCFA), saturated fatty acids, unsaturated or polyunsaturated fatty acids, branched fatty acids with polar or apolar functions, hydroxylated fatty acids, esters of fatty acids and iso aliphatic chain fatty acids, lipids including glycolipids, glycero-glycolipids, phospholipids or glycerophospholipids, long chain lipids, sphingolipids, sterids and cerides, unsaponifiable lipids such as terpenes, steroids (including sterols: phytosterols, cholesterols, vitamin D and other vitamins) and prostaglandins, esters, alkanes, alkenes, aldehydes, ketones, organic acids, antioxidants, polysaccharides, pigments, amino acids, MAAs, pseudopeptidic polymers of amino acids such as cyanophycin, enzymes or vitamins, antibiotics, compounds with a pharmacological activity, toxins, exogenous or recombinant proteins, organic compounds such as acids, molecules which can be used as biofuels, terpenes such as botryococcenes, or intermediate products that can be converted to fuel. Molecules used in order to produce bioplastics such as hydroxubutyrate (PHB) can also be mentioned.

In particular, this method has the advantage of enriching the thus-cultured cells with polyunsaturated fatty acids, more particularly, with eicosapentaenoic acid (EPA) and/or docosahexaenoic acid (DHA) and/or with arachidonic acid (ARA), and/or with a-linolenic acid (ALA), and/or with linoleic acid, and/or with oleic acid, and/or with carotenoids, more particularly, with lutein, canthaxanthin, astaxanthin, fucoxanthin, zeaxanthin, echinenone, beta-carotene and phoenicoxanthin.

The cell types which can be cultured according to the method described in this invention, preferably, according to embodiments comprising step a) and/or b) in mixotrophic mode, correspond to all the procaryotic or eucaryotic, photosensitive unicellular organisms capable of growing in mixotrophic culture. Moreover, it is desirable that the ability to grow in mixotrophic mode makes it possible to attain productivities in terms of biomass or molecules of interest compatible with industrial exploitation. By “photosensitive” is meant cells capable of inducing a metabolic activity in response to a natural or artificial illumination, under mixotrophic conditions. This metabolic activity can be for example a photosynthesis activity or the synthesis of a pigment.

The cells which can be cultured in the method according to the invention, preferably, according to embodiments comprising step a) and/or b) in mixotrophic mode, can be chosen from photosensitive eucaryotic cells isolated from an animal, plant or fungal, multicellular organism or photosensitive eucaryotic or procaryotic unicellular organisms.

Among the procaryotic microorganisms which can be cultured according to the method of the invention, there can be mentioned, non-limitatively, for example, the cyanobacteria, in particular the species Nostoc sp. which synthesizes exo-polysaccharides, or also marine bacteria which synthesize the molecules of industrial interest such as lipids, polysaccharides or pigments [Stafsnes M H et al.; J Microbiol. February 2010; 48(1):16-23].

As eucaryotic microorganisms, there can be mentioned, non-limitatively, marine and fresh-water protists, yeasts or cyanobacteria. These microorganisms can be photosynthetic or non-photosynthetic. Among the non-photosynthetic protists, there can be mentioned, in particular, those of the Labyrinthulomycetes class, in particular of the genus Schizochytrium or Aurantiochytrium, which are cultured under mixotrophic conditions for the simultaneous production of docosahexaenoic acid (DHA) and of carotenoid pigments such as astaxanthin or canthaxanthin. The protist Nitzschia brevirostris is a eucaryotic photosynthetic microorganism which is cultured under mixotrophic conditions for the simultaneous production of eicosapentaenoic acid (EPA) and of carotenoid pigments. Other photosensitive eucaryotic microorganisms such as yeasts can also be cultured using the method according to the invention, preferably, according to the embodiments comprising step a) and/or b) in mixotrophic mode.

As photosensitive eucaryotic cells, it is also possible to use, for example, eucaryotic cells isolated from a multicellular organism and capable of growing in mixotrophic culture. This type of organism can be fungal, animal or plant. The light supply during the growth and production phases makes it possible to maintain photosynthetic activity or photo-active metabolism, such as for example activation of the biosynthetic pathway of a pigment induced by light. Among the eucaryotic cells originating from multicellular organisms, there can in particular be mentioned the undifferentiated plant cells originating from calli which are maintained in this state by the addition of plant hormones to the culture medium; or also animal cells which have been treated by an immortalization process allowing in vitro culture.

Mixotrophic protists of interest that can be utilized in the method according to the invention can be, for example, selected from the species of the following genera: Schizochytrium, Thraustochytrium, Odontella, Phaeodactylum, Nanochloris, Crypthecodinium, Monodus, Nannochloropsis, Isochrysis, Euglena, Cyclotella, Nitzschia, Aurantiochytrium, Scenedesmus and/or Tetraselmis, Chlorella and Haematococcus.

According to a preferred aspect, the protists of interest are chosen from the species of the genera Schizochytrium, Thraustochytrium, Odontella, Phaeodactylum, Nanochloris, Crypthecodinium, Monodus, Nannochloropsis, Isochrysis, Euglena, Cyclotella, Nitzschia, Aurantiochytrium, Scenedesmus, and in particular from the species of the genera Schizochytrium, Thraustochytrium, Odontella, Phaeodactylum, Nanochloris, Crypthecodinium, Monodus, Nannochloropsis, Chlorella and Haematococcus.

Regarding the carotenoids, the following protists are particularly advantageous: Schizochytrium (astaxanthin), Nitzschia (fucoxanthin), Aurantiochytrium (canthaxanthin or astaxanthin) and Scenedesmus (lutein).

Tables 3A and 3B indicate novel strains identified by the inventors, which were deposited at the CCAP (Culture Collection of Algae and Protozoa, Scottish Association for Marine Science, Dunstaffnage Marine Laboratory, Oban, Argyll PA371QA, Scotland, United Kingdom) on 8 Jul. 2014, according to the provisions of the Treaty of Budapest, under the CCAP accession numbers indicated in these tables. The molecules produced by the strains are also indicated. Thus, the invention relates to these strains.

These genera form two groups: Group 1 is constituted by the strains belonging to the genera Schizochytrium, Aurantiochytrium and Crypthecodinium. These protists have no chloroplasts and are therefore called “non-photosynthetic”. Schizochytrium and Aurantiochytrium are unicellular fungi, and Crypthecodinium is a heterotrophic flagellate. Examples of these genera are given in Table 3A below and indicated by Group 1. According to an embodiment, the method of the invention relates to the culture of the genera Schizochytrium, Aurantiochytrium and Crypthecodinium.

The second group, Group 2, is constituted by the strains which are photo-synthetic, i.e. having chloroplasts. These genera of protists Tetraselmis, Scenedesmus, Chlorella, Nitzschia and Haematococcus are classed as microalgae. According to an embodiment, the method of the invention relates to the culture of the genera Tetraselmis, Scenedesmus, Chlorella, Nitzschia and Haematococcus.

Group 1

TABLE 3A
Examples of the strains which can be utilized according to the
method of the invention. These strains are protists called non-photosynthetic,
i.e. having no chloroplasts. According to an embodiment, the method of the
invention relates to the culture belonging to the genera Schizochytrium,
Aurantiochytrium and Crypthecodinium.

| | CCAP deposit | |
|---|---|---|
| Strains | No. | Molecule of interest |
| Schizochytrium sp. FCC 36 | CCAP 4087/3 | DHA, astaxanthin |
| Schizochytrium sp. FCC1320 | CCAP 4087/4 | DHA, astaxanthin |
| Schizochytrium sp. FCC1491 | CCAP 4087/5 | DHA, astaxanthin |
| Aurantiochytrium mangrovei | CCAP 4062/3 | DHA, canthaxanthin, |
| FCC 1311 | | astaxanthin |
| Aurantiochytrium mangrovei | CCAP 4062/2 | DHA, canthaxanthin, |
| FCC 31 | | astaxanthin |
| Aurantiochytrium mangrovei | CCAP 4062/4 | DHA, canthaxanthin, |
| FCC 1319 | | astaxanthin |
| Aurantiochytrium mangrovei | CCAP 4062/6 | DHA, canthaxanthin, |
| FCC 1479 | | astaxanthin |
| Aurantiochytrium mangrovei | CCAP 4062/5 | DHA, canthaxanthin, |
| FCC 1325 | | astaxanthin |
| Crypthecodinium cohnii | CCAP 1104/3 | DHA, carotenoids (β- |
| FCC 30 | | carotene) |
| Crypthecodinium cohnii | CCAP 1104/5 | DHA, carotenoids (β- |
| FCC 1384 | | carotene) |
| Crypthecodinium cohnii | CCAP 1104/4 | DHA, carotenoids (β- |
| FCC 1348 | | carotene) |

Group 2

TABLE 3B
Examples of the strains which can be utilized according to the
method of the invention. These strains are microalgae
called photosynthetic, i.e. having chloroplasts.
These genera are microalgae. According to an
embodiment, the method of the invention
relates to the culture of the genera Tetraselmis,
Scenedesmus, Chlorella, Nitzschia and Haematococcus.

| Strains | CCAP deposit No. | Molecule of interest |
|---|---|---|
| Tetraselmis sp. FCC 1563 | CCAP 66/85 | EPA, ALA |
| Scenedesmus abundans | CCAP 276/78 | ALA, oleic acid, |
| FCC 23 | | lutein |
| Scenedesmus sp. FCC 1483 | CCAP 276/79 | ALA, oleic acid, |
| | | lutein |
| Scenedesmus obliquus FCC 4 | CCAP 276/77 | ALA, oleic acid, |
| | | lutein |
| Chlorella sorokiniana FCC 2 | CCAP 211/129 | lutein |
| Chlorella sp. FCC 1553 | CCAP 211/130 | lutein |
| Chlorella sp. FCC 1520 | CCAP 211/131 | lutein |
| Nitzschia sp. FCC 1687 | CCAP 1052/22 | EPA, fucoxanthin |
| Haematococcus sp. FCC | CCAP 34/18 | astaxanthin |
| 1643 | | |

The invention also relates to the novel strains of Tables 3A and 3B. These strains have been selected for their mixotrophic character and for their high yield of fatty acids and/or of carotenoids, in particular of lutein, fucoxanthin, astaxanthin, canthaxanthin and β-carotene, and in particular for their ability to be cultured with a light supply greater than 10 μE, in a medium rich in organic elements. These media are known to one skilled in the art.

The culture method according to the invention can also apply to any species of the genus Schizochytrium, Aurantiochytrium, Crypthecodinium, Tetraselmis, Scenedesmus, Chlorella, Nitzschia and Haematococcus capable of growing under mixotrophic conditions according to the invention, and capable of producing fatty acids and/or carotenoids.

When the microalgae are of the genus Chloralla, they can be chosen from the species C. acuminata, C. angustoellipsoidea, C. anitrata, C. antarctica, C. aureoviridis, C. autotrophica, C. botryoides, C. caldaria, C. candida, C. capsulata, C. chlorelloides, C. cladoniae, C. coelastroides, C. colonialis, C. communis, C. conductrix, C. conglomerata, C. desiccata, C. effipsoidea, C. elongata, C. emersonii, C. faginea, C. fusca, C. glucotropha, C. homosphaera, C. infusionum, C. kessleri, C. koettlitzii, C. lacustris, C. lewinii, C. lichina, C. lobophora, C. luteo-viridis, C. marina, C. miniata, C. minor, C. minutissima, C. mirabilis, C. mucosa, C. mutabilis, C. nocturna, C. nordstedtii, C. oblonga, C. oocystoides, C. ovalis, C. paramecii, C. parasitica, C. parva, C. peruviana, C. photophila, C. pituita, C. pringsheimii, C. protothecoides, C. pulchelloides, C. pyrenoidosa, C. regularis, C. reisiglii, C. reniformis, C. rotunda, C. rubescens, C. rugosa, C. saccharophila, C. salina, C. simplex, C. singularis, C. sorokiniana, C. spaerckii, C. sphaerica, C. stigmatophora, C. subsphaerica, C. terricola, C. trebouxioides, C. vannielii, C. variabilis, C. viscosa, C. volutis, C. vulgaris, C. zopfingiensis. Advantageously according to the invention the algae of the genus Chlorella can be algae chosen from the species C. sorokiniana or C. vulgaris.

When the microalgae are of the genus Scenedesmus, they can be chosen from the species S. abundans, S. aciculatus, S. aculeolatus, S. aculeotatus, S. acuminatus, S. acutiformis, S. acutus, S. aldavei, S. alternans, S. ambuehlii, S. anhuiensis, S. anomalus, S. antennatus, S. antillarum, S. apicaudatus, S. apiculatus, S. arcuatus, S. aristatus, S. armatus, S. arthrodesmiformis, S. arvernensis, S. asymmetricus, S. bacillaris, S. baculiformis, S. bajacalifomicus, S. balatonicus, S. basiliensis, S. bernardii, S. bicaudatus, S. bicellularis, S. bidentatus, S. bijuga, S. bijugatus, S. bijugus, S. brasiliensis, S. breviaculeatus, S. brevispina, S. caribeanus, S. carinatus, S. caudato-aculeolatus, S. caudatus, S. chlorelloides, S. circumfusus, S. coalitus, S. costatogranulatus, S. crassidentatus, S. curvatus, S. decorus, S. denticulatus, S. deserticola, S. diagonalis, S. dileticus, S. dimorphus, S. disciformis, S. dispar, S. distentus, S. ecornis, S. effipsoideus, S. effipticus, S. falkatus, S. fenestratus, S. flavescens, S. flexuosus, S. furcosus, S. fuscus, S. fusiformis, S. gracilis, S. graevenitzii, S. grahneisii, S. granulatus, S. gujaratensis, S. gutwinskii, S. hanleyi, S. helveticus, S. heteracanthus, S. hindakii, S. hirsutus, S. hortobagyi, S. houlensis, S. huangshanensis, S. hystrix, S. incrassatulus, S. indianensis, S. indicus, S. inermis, S. insignis, S. intermedius, S. javanensis, S. jovais, S. jugalis, S. kerguelensis, S. kissii, S. komarekii, S. lefevrei, S. linearis, S. littoralis, S. longispina, S. longus, S. luna, S. lunatus, S. magnus, S. maximus, S. microspina, S. minutus, S. mirus, S. morzinensis, S. multicauda, S. multiformis, S. multispina, S. multistriatus, S. naegelii, S. nanus, S. notatus, S. nygaardii, S. oahuensis, S. obliquus, S. obtusiusculus, S. obtusus, S. olvaltemus, S. oocystiformis, S. opoliensis, S. ornatus, S. ovaltemus, S. pannonicus, S. papillosum, S. parisiensis, S. parvus, S. pecsensis, S. pectinatus, S. perforatus, S. planctonicus, S. plarydiscus, S. platydiscus, S. pleiomorphus, S. polessicus, S. polydenticulatus, S. polyglobulus, S. polyspinosus, S. praetervisus, S. prismaticus, S. producto-capitatus, S. protuberans, S. pseudoarmatus, S. pseudobemardii, S. pseudodenticulatus, S. pseudo granulatus, S. pseudohystrix, S. pyrus, S. quadrialatus, S. quadricauda, S. quadricaudata, S. quadricaudus, S. quadrispina, S. raciborskii, S. ralfsii, S. reginae, S. regularis, S. reniformis, S. rostrato-spinosus, S. rotundus, S. rubescens, S. scenedesmoides, S. schnepfii, S. schroeteri, S. securiformis, S. semicristatus, S. semipulcher, S. sempervirens, S. senilis, S. serrato-perforatus, S. serratus, S. serrulatus, S. setiferus, S. sihensis, S. smithii, S. soli, S. sooi, S. spicatus, S. spinoso-aculeolatus, S. spinosus, S. spinulatus, S. striatus, S. subspicatus, S. tenuispina, S. terrestris, S. tetradesmiformis, S. transilvanicus, S. tricostatus, S. tropicus, S. tschudyi, S. vacuolatus, S. variabilis, S. velitaris, S. verrucosus, S. vesiculosus, S. westii, S. weberi, S. wisconsinensis, S. wuhanensis, S. wuhuensis. Advantageously according to the invention the algae of the genus Scenedesmus can be algae chosen from the species S. obliquus or S. abundans.

When the microalgae are of the genus Nitzschia, N. abbreviata, N. abonuensis, N. abridia, N. accedens, N. accommodata, N. aciculariformis, N. acicularioides, N. acicularis (comprising all these varieties), N. acidoclinata, N. actinastroides, N. actydrophila, N. acuta, N. acuminata (comprising all these varieties), N. acuta, N. adamata, N. adamatoides, N. adapta, N. adducta, N. adductoides, N. admissa, N. admissoides, N. aequalis, N. aequatorialis, N. aequora, N. aequorea, N. aerophila, N. aerophiloides, N. aestuari, N. affinis, N. africana, N. agnewii, N. agnita, N. alba, N. albicostalis, N. alexandrina, N. alicae, N. allanssonii, N. alpina, N. alpinobacillum, N. amabilis, N. ambigua, N. americana, N. amisaensis, N. amphibia, N. amphibia (comprising all these varieties), N. amphibioides, N. amphicephala, N. amphilepta, N. amphioxoides, N. amphioxys (comprising all these varieties), N. amphiplectans, N. amphiprora, N. amplectens, N. amundonii, N. anassae, N. andicola, N. angularis (comprising all these varieties), N. angu/ata, N. angustata (comprising all these varieties), N. angustatula, N. angustiforaminata, N. aniae, N. antarctica, N. antillarum, N. apiceconica, N. apiculata, N. archibaldii, N. arcuata, N. arcula, N. arcus, N. ardua, N. aremonica, N. arenosa, N. areolata, N. armoricana, N. asperula, N. astridiae, N. atomus, N. attenuata, N. aurantiaca, N. aurariae, N. aurica, N. auricula, N. australis, N. austriaca, N. bacata (comprising all these varieties), N. bacillariaeformis, N. bacilliformis, N. bacillum, N. balatonis, N. balkanica, N. baltica, N. barbieri (comprising all these varieties), N. barkleyi, N. barronii, N. barrowiana, N. bartholomei, N. bathurstensis, N. bavarica, N. behrei, N. bergii, N. beyeri, N. biacrula, N. bicapitata (comprising all these varieties), bicuneata, N. bifurcata, N. bilobata (comprising all these varieties), N. birostrata, N. bisculpta, N. bita, N. bizertensis, N. blankaartensis, N. bombiformis, N. borealis, N. bosumtwiensis, N. braarudii, N. brebissonii (comprising all these varieties), N. bremensis (comprising all these varieties), N. brevior, N. brevirostris, N. brevissima (comprising all these varieties), N. brevistriata, N. brightwellii, N. brittonii, N. brunoi, N. bryophila, N. buceros, N. bukensis, N. bulnheimiana, N. buschbeckii, N. calcicola, N. caledonensis, N. calida (comprising all these varieties), N. califomica, N. campechiana, N. capensis, N. capitata, N. capitellata (comprising all these varieties), N. capuluspalae, N. carnicobarica, N. carnico-barica, N. challengeri, N. chalonii, N. chandolensis, N. chardezii, N. chasei, N. chauhanii, N. chungara, N. chutteri, N. circumsuta, N. clarissima, N. clausii, N. clementei, N. clementia, N. clevei, N. closterium (comprising all these varieties), N. coarctata, N. cocconeiformis, N. communis (comprising all these varieties),N. commutata, N. commutatoides, N. compacta, N. compressa (comprising all these varieties), N. concordia, N. confinis, N. conformata, N. confusa, N. congolensis, N. constricta (comprising all these varieties), N. consummata, N. corpulenta, N. costei, N. costei, N. creticola, N. cucumis, N. cursoria, N. curta, N. curvata, N. curvilineata, N. curvipunctata, N. curvirostris (comprising all these varieties), N. curvula (comprising all these varieties), N. cuspidata, N. cylindriformis, N. cylindrus, N. dakariensis, N. davidsonii, N. dealpina, N. debilis, N. decipiens, N. delauneyi, N. delicatissima, N. delicatula, N. delognei, N. denticula (comprising all these varieties), N. denticuloides, N. desertorum, N. dianae, N. diaphana, N. diducta, N. didyma, N. dietrichii, N. dilatata, N. diluviana, N. dippelii, N. directa, N. diserta, N. disputata, N. dissipata (comprising all these varieties), N. dissipatoides, N. distans (comprising all these varieties), N. distantoides, N. divaricata, N. divergens, N. diversa, N. diversecostata, N. doljensis, N. draveillensis, N. droebakensis, N. dubia (comprising all these varieties), N. dubiformis, N. dubioides, N. ebroicensis, N. eglei, N. elegans, N. elegantula, N. elegens, N. elliptica, N. elongata, N. entomon, N. epiphytica, N. epiphyticoides, N. epithemiformis, N. epithemioides, N. epithemoides (comprising all these varieties), N. epsilon, N. erlandssonii, N. erosa, N. etoshensis, N. examinanda, N. eximia, N. famelica, N. fasciculata, N. febigeri, N. ferox, N. ferrazae, N. fibula-fissa, N. filiformis (comprising all these varieties), N. flexa, N. flexoides, N. fluminensis, N. fluorescens, N. fluvialis, N. fogedii, N. fonticola (comprising all these varieties), N. fonticoloides, N. fonticula, N. fontifuga, N. forfica, N. formosa, N. fossalis, N. fossilis, N. fragilariiformis, N. franconica, N. fraudulenta, N. frauenfeldii, N. frequens, N. frickei, N. frigida (comprising all these varieties), N. frustuloides, N. frustulum (comprising all these varieties), N. fruticosa, N. fundi, N. fusiformis, N. gaarderi, N. gaertnerae, N. gandersheimiensis, N. garrensis, N. gazellae, N. geitleri, N. geitlerii, N. gelida (comprising all these varieties), N. geniculata, N. gessneri, N. gieskesii, N. gigantea, N. gisela, N. glabra, N. glacialis (comprising all these varieties), N. glandiformis, N. goetzeana (comprising all these varieties), N. gotlandica, N. graciliformis, N. gracilis (comprising all these varieties), N. gracillima, N. graciloides, N. gradifera, N. graeffii, N. grana, N. grandis, N. granii (comprising all these varieties), N. granulata (comprising all these varieties), N. granulosa, N. groenlandica, N. grossestriata, N. grovel, N. gruendleri, N. grunowii, N. guadalupensis, N. guineensis, N. guttula, N. gyrosigma, N. habirshawii, N. habishawii, N. hadriatica, N. halteriformis, N. hamburgiensis, N. hantzschiana (comprising all these varieties), N. harderi, N. harrissonii, N. hassiaca, N. heidenii, N. heimii, N. hemistriata, N. heteropolica, N. heuflerania, N. heufleriana (comprising all these varieties), N. hiemalis, N. hiengheneana, N. hierosolymitana, N. hoehnkii, N. holastica, N. hollerupensis, N. holsatica, N. homburgiensis, N. hudsonii, N. hummii, N. hungarica (comprising all these varieties), N. hustedti, N. hustedtiana, N. hyalina, N. hybrida (comprising all these varieties), N. hybridaeformis, N. ignorata (comprising all these varieties), N. iltisii, N. impressa, N. improvisa, N. incerta, N. incognita, N. inconspicua, N. incrustans, N. incurva (comprising all these varieties), N. indica, N. indistincta, N. inducta, N. inflatula, N. ingenua, N. inimasta, N. innominata, N. insecta, N. insignis (comprising all these varieties), N. intermedia (comprising all these varieties), N. intermissa, N. interrupta, N. interruptestriata, N. invicta (comprising all these varieties), N. in visa, N. invisitata, N. iranica, N. irregularis, N. irremissa, N. irrepta, N. irresoluta, N. irritans, N. italica, N. janischii, N. jelineckii, N. johnmartinii, N. juba, N. jucunda, N. jugata (comprising all these varieties), N. jugiformis, N. kahlii, N. kanakarum, N. kanayae, N. kavirondoensis, N. kerguelensis, N. kimberliensis, N. kittlii, N. kittonii, N. knysnensis, N. kolaczeckii, N. kotschyi, N. kowiensis, N. krachiensis, N. krenicola, N. kuetzingiana (comprising all these varieties), N. kuetzingii, N. kuetzingioides, N. kurzeana, N. kurzii, N. kützingiana (comprising all these varieties), N. labella, N. labuensis, N. lacrima, N. lacunarum, N. lacunicola, N. lacus-karluki, N. lacustris, N. lacuum, N. laevis, N. laevissima, N. lagunae, N. lagunensis, N. lamprocampa (comprising all these varieties), N. lanceola (comprising all these varieties), N. lanceolata (comprising all these varieties), N. lancettula, N. lancettuloides, N. lange-bertalotii, N. latens, N. latestriata, N. latiuscula, N. lauenbergiana, N. lauenburgiana, N. lecointei, N. leehyi, N. legleri, N. lehyi, N. leistikowii, N. lesbia, N. lesinensis, N. lesothensis, N. leucosigma, N. levidensis (comprising all these varieties), N. liebetruthii (comprising all these varieties), N. ligowskii, N. limicola, N. limulus, N. linearis (comprising all these varieties), N. lineata, N. lineola, N. linkei, N. lionella, N. littoralis (comprising all these varieties), N. littorea, N. longa, N. longicollum, N. longirostris, N. longissima (comprising all these varieties), N. lorenziana (comprising all these varieties), N. lucisensibilis, N. lunaris, N. lunata, N. lurida, N. luzonensis, N. macaronesica, N. macedonica, N. macera, N. machardyae, N. macilenta (comprising all these varieties), N. magnacarina, N. mahihaensis, N. mahoodii, N. maillardii, N. major, N. majuscula (comprising all these varieties), N. makarovae, N. manca, N. mancoides, N. manguini, N. marginata, N. marginulata (comprising all these varieties), N. marina, N. martiana, N. maxima, N. media, N. medioconstricta, N. mediocris, N. mediterranea, N. metzeltinii, N. microcephala (comprising all these varieties), N. migrans, N. minuta, N. minutissima, N. minutula, N. miramarensis, N. miserabilis, N. mitcheffiana, N. modesta, N. moissacensis (comprising all these varieties), N. mollis, N. monachorum, N. monoensis, N. montanestris, N. morosa, N. multistriata, N. nana, N. natalensis, N. natans, N. nathorsti, N. navicularis, N. navis-varingica, N. navrongensis, N. neglecta, N. nelsonii, N. neocaledonica, N. neoconstricta, N. neofrigida, N. neogena, N. neotropica, N. nereidis, N. nicobarica, N. nienhuisii, N. normannii, N. notabilis, N. nova, N. novae-guineaensis, N. novae-guineensis, N. novaehollandiae, N. nova-zealandia, N. nyassensis, N. oberheimiana, N. obesa, N. obliquecostata, N. obscura, N. obscurepunctata, N. obsidialis, N. obsoleta, N. obsoletiformis, N. obtusa (comprising all these varieties), N. obtusangula, N. oceanica, N. ocellata, N. oliffi, N. omega, N. osmophila, N. ossiformis, N. ostenfeldii, N. ovalis, N. paaschei, N. pacifica, N. palacea, N. palea (comprising all these varieties), N. paleacea, N. paleaeformis, N. paleoides, N. palustris, N. pamirensis, N. panduriformis (comprising all these varieties), N. pantocsekii, N. paradoxa (comprising all these varieties), N. parallela, N. pararostrata, N. partita, N. parvula (comprising all these varieties), N. parvuloides, N. paxillifer, N. peisonis, N. pelagica, N. pellucida, N. pennata, N. peragaffii, N. perindistincta, N. perminuta, N. perpusilla (comprising all these varieties), N. perspicua, N. persuadens, N. pertica, N. perversa, N. petitiana, N. philippinarum, N. pilum, N. pinguescens, N. piscinarum, N. plana (comprising all these varieties), N. planctonica, N. plicatula, N. plioveterana, N. polaris, N. polymorpha, N. ponciensis, N. praecurta, N. praefossilis, N. praereinholdii, N. princeps, N. procera, N. prolongata (comprising all these varieties), N. prolongatoides, N. promare, N. propinqua, N. pseudepiphytica, N. pseudoamphioxoides, N. pseudoamphioxys, N. pseudoamphyoxys, N. pseudoatomus, N. pseudobacata, N. pseudocapitata, N. pseudocarinata, N. pseudocommunis, N. pseudocylindrica, N. pseudodelicatissima, N. pseudofonticola, N. pseudohungarica, N. pseudohybrida, N. pseudonana, N. pseudoseriata, N. pseudosigma, N. pseudosinuata, N. pseudostagnorum, N. pubens, N. pulcherrima, N. pumila, N. punctata (comprising all these varieties), N. pungens (comprising all these varieties), N. pungiformis, N. pura, N. puriformis, N. pusilla (comprising all these varieties), N. putrida, N. quadrangula, N. quickiana, N. rabenhorstii, N. radicula (comprising all these varieties), N. rautenbachiae, N. recta (comprising all these varieties), N. rectiformis, N. rectilonga, N. rectirobusta, N. rectissima, N. regula, N. reimeri, N. reimerii, N. reimersenii, N. retusa, N. reversa, N. rhombica, N. rhombiformis, N. rhopalodioides, N. richterae, N. rigida (comprising all these varieties), N. ritscheri, N. robusta, N. rochensis, N. rolandii, N. romana, N. romanoides, N. romanowiana, N. rorida, N. rosenstockii, N. rostellata, N. rostrata, N. ruda, N. rugosa, N. rupestris, N. rusingae, N. ruttneri, N. salinarum, N. salinicola, N. salpaespinosae, N. salvadoriana, N. sansimoni, N. sarcophagum, N. scabra, N. scalaris, N. scaligera, N. scalpelliformis, N. schoenfeldii, N. schwabei, N. schweikertii, N. scutellum, N. sellingii, N. semicostata, N. semirobusta, N. separanda, N. seriata (comprising all these varieties), N. serpenticola, N. serpentiraphe, N. serrata, N. sibula (comprising all these varieties), N. sigma (comprising all these varieties), N. sigmaformis, N. sigmatella, N. sigmoidea (comprising all these varieties), N. silica, N. silicula (comprising all these varieties), N. siliqua, N. similis, N. simplex, N. simpliciformis, N. sinensis, N. sinuata (comprising all these varieties), N. smithii, N. sociabilis, N. socialis (comprising all these varieties), N. solgensis, N. solida, N. solita, N. soratensis, N. sp., N. spathulata (comprising all these varieties), N. speciosa, N. spectabilis (comprising all these varieties), N. sphaerophora, N. spiculoides, N. spiculum, N. spinarum, N. spinifera, N. stagnorum, N. steenbergensis, N. stellata, N. steynii, N. stimulus, N. stoliczkiana, N. stompsii (comprising all these varieties), N. strelnikovae, N. stricta, N. strigillata, N. striolata, N. subaccommodata, N. subacicularis, N. subacuta, N. subamphioxioides, N. subapiculata, N. subbacata, N. subcapitata, N. subcapitellata, N. subcohaerens (comprising all these varieties), N. subcommunis, N. subconstricta, N. subcurvata, N. subdenticula, N. subfalkata, N. subfraudulenta, N. subfrequens, N. subfrustulum, N. subgraciloides, N. subinflata, N. subinvicta, N. sublaevis, N. sublanceolata, N. sublica, N. sublinearis, N. sublongirostris, N. submarina, N. submediocris, N. subodiosa, N. subpacifica, N. subpunctata, N. subromana, N. subrostrata, N. subrostratoides, N. subrostroides, N. subsalsa, N. subtilioides, N. subtilis (comprising all these varieties), N. subtubicola, N. subvitrea, N. suchlandtii, N. sulcata, N. sundaensis, N. supralitorea, N. tabellaria, N. taenia, N. taeniiformis, N. tantata, N. tarda, N. taylorii, N. temperei, N. tenella, N. tenerifa, N. tenuiarcuata, N. tenuirostris, N. tenuis (comprising all these varieties), N. tenuissima, N. tergestina, N. terrestris, N. terricola, N. thermalis (comprising all these varieties), N. thermaloides, N. tibetana, N. tirstrupensis, N. tonoensis, N. towutensis, N. translucida, N. tropica, N. tryblionella (comprising all these varieties), N. tsarenkoi, N. tubicola, N. tumida, N. turgidula, N. turgiduloides, N. umaoiensis, N. umbilicata, N. umbonata, N. vacillata, N. vacua, N. valdecostata, N. valdestriata, N. valens, N. valga, N. valida (comprising all these varieties), N. vanheurckii, N. vanoyei, N. vasta, N. ventricosa, N. vermicularioides, N. vermicularis (comprising all these varieties), N. vermicularoides, N. vexans, N. victoriae, N. vidovichii, N. vildaryana, N. villarealii, N. virgata, N. visurgis, N. vitrea (comprising all these varieties), N. vivax (comprising all these varieties), N. vixnegligenda, N. vonhauseniae, N. vulga, N. weaveri, N. weissflogii, N. westii, N. williamsiii, N. wipplingeri, N. witkowskii, N. wodensis, N. woltereckii, N. woltereckoides, N. wuellerstorfii, N. wunsamiae, N. yunchengensis, N. zebuana, N. zululandica.

Advantageously according to the invention, the algae of the genus Nitzschia can be algae chosen from the species N. sp.

When the microalgae are of the genus Haematococcus, they can be chosen from the species H. allmanii, H. buetschlii, H. capensis, H. carocellus, H. droebakensis, H. grevilei, H. insignis, H. lacustris, H. murorum, H. pluvialis, H. salinus, H, sanguineis, H. thermalis, H. zimbabwiensis.

When the microalgae are of the genus Aurantiochytrium, they can be chosen from the species: A. limacinum, A. mangrovei.

When the microalgae are of the genus Schizochytrium, they can be chosen from the species: S. aggregatum, S. limacinum, S. mangrovei, S. minutum, S. octosporum.

When the microalgae are of the genus Crypthecodinium, they can be chosen from the species: C. cohnii, C. setense.

When the microalgae are of the genus Tetraselmis, they can be chosen from the species: T. alacris, T. apiculata, T. amoldii, T. ascus, T. astigmatica, T. bichlora, T. bilobata, T. bolosiana, T. chui, T. contracta, T. convolutae, T. cordiformis, T. desikacharyi, T. elliptica, T. fontiana, T. gracilis, T. hazenii, T. helgolandica, T. impellucida, T. incisa, T. inconspicua, T. indica, T. levis, T. maculata, T. marina, T. mediterranea, T. micropapillata, T. rubens, T. striata, T. subcordiformis, T. suecica, T. tetrabrachia, T. tetrathele, T. verrucosa, T. viridis, T. wettsteinii.

According to an embodiment of the invention, the culture of filamentous fungi can be carried out in aerated fermenters and with mechanical stirring such as those described in the invention. However, the culture is carried out under appropriate stirring conditions known to one skilled in the art, limiting the effects of shearing and allowing culture under mixotrophic conditions of the cells in the form of filaments or isolated cells. The effects of light on the metabolism of these organisms are known and the metabolites of industrial interest such as pigments can be produced during culture under mixotrophic conditions [Folia Microbiol (Praha). 2013 Apr. 2. Light regulation on growth, development, and secondary metabolism of marine-derived filamentous fungi. Cai M, Fang Z, Niu C, Zhou X, Zhang Y.]

The genera of fungi which can be cultured according to the method of the invention are by way of example Mortierella alpina for the production of ARA or Aspergillus niger for the production of citric acid.

The protist Aurantiochytrium can be cultured according to the method of the invention for the production of DHA. Preferably, the culture is carried out according to a preferred embodiment of the invention, in which steps a) and b) are carried out under mixotrophic conditions. By way of example, the culture of the strain Aurantiochytrium Mangrovei FCC 1324, a strain isolated by the presents inventors and deposited at the CCAP (Culture Collection of Algae and Protozoa, Scottish Association for Marine Science, Dunstaffnage Marine Laboratory, Oban, Argyll PA371 QA, Scotland, United Kingdom) according to the provisions of the Treaty of Budapest, under the accession number CCAP 4062/1, on 21 Jun. 2013, according to the method of the invention can make it possible to produce a biomass rich in DHA and in astaxanthin and/or canthaxanthin. Said DHA can represent more than 40%, or more than 50%, or more than 60% of the total lipids contained in the protist, the carotenoids being rich in astaxanthin and/or canthaxanthin, and said astaxanthin and/or canthaxanthin can represent more than 0.1%, or more than 0.15%, or more than 0.2% by weight relative to the total weight of dry matter. The strain can attain a level of productivity (quantity of product of interest produced, per litre of culture, per hour) of 0.015 mg/L/h, or more than 0.020 mg/L/h, or more than 0.025 mg/L/h (see Table 2 in Example 1).

The culture of plant cells can be envisaged according to an embodiment of the invention such as, for example, cell suspensions of the plant Adonis annua belonging to the Ranunculaceae family. Cultured under mixotrophic conditions, Adonis annua is capable of producing astaxanthin and adonirubin (intermediate metabolite of the biosynthesis of astaxanthin from beta-carotene), which has anti-oxidant and anti-tumoral activity in humans.

The yeast Rhodotorula glutinis, according to the method of the invention, has an industrial interest because of its growth rate, its richness in oil and the synthesis of a carotenoid pigment, beta-carotene. The culture of Rhodotorula glutinis can be envisaged advantageously according to the method of the invention as the illumination has been shown to be as beneficial for growth [Yen H W, Zhang Z.; Bioresource Technology. October 2011; 102(19):9279-81] and also for the synthesis of pigments [Bhosale P, Gadre R V.; Lett Appl Microbiol. 2002; 34(5):349-53]. The uncoupled culture makes it possible to apply physico-chemical and/or illumination parameters which are orientated towards the growth of the cells, then in a second phase, towards the accumulation of oil and/or of the pigment of interest.

A few advantages of the invention are illustrated in Table 5. Table 5 illustrates the results of a comparison for the production of biomass by Aurantiochytrium, then the extraction of oil rich in polyunsaturated fatty acids according to the method of the invention and the discontinuous, semi-continuous and continuous method. The method of the invention (uncoupled) makes it possible to combine the advantages of the continuous and semi-continuous (“Fed-Batch”) mode by limiting the phases of stopping production for cleaning and sterilization of equipment, while allowing a biomass of optimum composition to be obtained by means of the maturation phase.

Advantageously, the method according to the embodiment of the invention allows, for the culture of protists, yeasts or bacteria, the production of a biomass comprising from 40 g/L to 250 g/L of dry matter, and preferentially above 80 g/L. For example, Aurantiochytrium can produce 150 g/L as certain bacteria or yeasts.

Said protist biomass can also comprise a fatty matter content of at least 10%, preferably at least 20%, more preferentially at least 30%. For example, in the case of Aurantiochytrium, the fatty matter content of the biomass is at least 30%.

For example, the strains of the genus Schizochytrium sp, under mixotrophic conditions according to an embodiment of the invention, produce DHA and astaxanthin. They generally give a yield of biomass comprised between 80 and 200 g/L, with a level of lipids between 30 and 60% of the dry matter, in which docosahexaenoic acid (DHA) represents 40 to 60%. The yield for astaxanthin is generally from 0.01 to 0.2% of the dry matter. The strain FCC 36 can be mentioned as an example of these strains.

The strains of the genus Aurantiochytrium, under mixotrophic conditions according to an embodiment of the invention, produce DHA and astaxanthin and/or canthaxanthin. The strains generally give a yield of biomass comprised between 80 and 200 g/L, with a level of lipids of approximately 50% of the dry matter. Docosahexaenoic acid (DHA) generally represents between 15 to 50% of the fatty acids; astaxanthin and/or canthaxanthin generally represent(s) 0.01 to 0.2% of the dry matter. The strains of Aurantiochytrium mangrovei FCC 1311, FCC 1319, FCC 1325 and FCC 31 can be mentioned as examples of these strains.

The strains of the genus Crypthecodinium, in particular of the species Crypthecodinium cohnii, under mixotrophic conditions according to an embodiment of the invention, produce DHA and carotenoids, more particularly β-carotene. The strains generally give a yield of biomass comprised between 50 and 200 g/L, with a level of lipids between 10 and 30% in dry matter. Docosahexaenoic acid (DHA) generally represents between 15 to 50% of the fatty acids and carotenoids, more particularly β-carotene, represent between 0.01 to 0.2% of the dry matter. The strains Crypthecodinium cohnii FCC 1384, FCC 1348 and FCC 30 can be mentioned as examples of these strains.

The strains of the genus Chlorella, in particular Chlorella sp. and Chlorella sorokiniana produce lutein. The strains make it possible to produce, under mixotrophic conditions according to an embodiment of the invention, generally between 60 g/L and 150 g/L of biomass, and lutein at levels generally between 0.1 and 5% of the dry matter. The biomass thus produced is particularly suitable for feeding rotifers intended for fish farming, in particular for farming bass and sea bream. It is also used as a nutraceutical for its immunostimulating and detoxifying properties. The strains Chlorella FCC 2, the strains Chlorella sp. FCC 1553 and FCC 1520 can be mentioned as examples of these strains.

The strains of the genus Scenedesmus, in particular of the species Scenedesmus obliquus, Scenedesmus sp. and Scenedesmus abundans under mixotrophic conditions according to the invention, produce ALA, oleic acid and lutein. The strains generally give a yield of biomass comprised between 30 and 100 g/L, with a level of lipids generally between 10 and 60% of the dry matter. Alpha linoleic acid or ALA generally represents between 10 and 50% of the total fatty acids, and oleic acid generally represents between 25 and 50% of the total fatty acids, lutein generally represents between 0.1 to 5% of the dry matter. The strains Scenedesmus obliquus FCC 4, Scenedesmus sp. FCC 1483 and Scenedesmus abundans FCC 23 can be mentioned as examples of these strains.

The strains of the genus Tetraselmis, in particular Tetraselmis sp. produce, under mixotrophic conditions according to an embodiment of the invention, EPA and ALA. Generally, the strains give a yield of biomass between 30 and 80 g/L, with a level of fatty acids generally between 10 and 30% of the dry matter. Eicosapentaenoic acid (EPA) generally represents between 10 to 25% of the fatty acids, and a-linolenic acid (ALA), generally 5 to 20% of the fatty acids. The biomass thus produced is particularly suitable for use in aquaculture. The strain Tetraselmis sp. FCC 1563 can be mentioned as an example of these strains.

The strains of the genus Haematococcus produce, under mixotrophic conditions according to an embodiment of the invention, astaxanthin. The strains give a yield of biomass generally between 5 and 30 g/L, and of astaxanthin generally between 0.1 to 15% of the dry matter. The strain Haematococcus sp. FCC 1643 can be mentioned as an example of these strains.

The strains of the genus Nitzschia produce, under mixotrophic conditions according to an embodiment of the invention, EPA and fucoxanthin. The strains generally give a yield of biomass comprised between 40 and 120 g/L, a yield of lipids between 10 and 50% of the dry matter, of eicosapentaenoic acid (EPA), between 15 and 50% of the fatty acids and of fucoxanthin, between 0.1 and 5% of the dry matter. The strains Nitzschia sp. FCC 1687 can be mentioned as examples of these strains.

In the present invention, the strains which are cultured under mixotrophic conditions, in particular in the presence of a variable and/or discontinuous illumination, in particular in the form of flashes, making it possible to produce the molecules of interest, in particular lipids and/or pigments. Generally, the culture of these strains belonging to the genera Schizochytrium, Aurantiochytrium, Crypthecodinium, Scenedesmus and Nitzschia, which produce a lipid and a pigment under heterotrophic conditions, produce no or very little pigment. In addition, quantities of biomass obtained in mixotrophic mode for these strains, according to certain embodiments of the invention, are equal to or even greater (for example, approximately 10 to 18% greater) than the quantities obtained under heterotrophic conditions. By “heterotrophic conditions”, is meant culture conditions with an identical culture medium, but in the absence of light.

The invention thus relates to a method for the culture of protists, in mixotrophic mode, in particular in the presence of a variable or discontinuous illumination over time, for example in the form of flashes, in particular with a view to producing poly-unsaturated or mono-unsaturated fatty acids, and/or carotenoids, in particular lutein, fucoxanthin, astaxanthin, canthaxanthin and β-carotene.

According to a preferred aspect, said biomass comprises a content of fatty acid(s) of interest in the fatty phase of at least 10%, preferably at least 20%, more preferentially at least 30%.

Said biomass can also comprise a content of pigment(s) of interest in the fatty phase of at least 0.01%, preferably at least 0.1%, more preferentially at least 0.5%.

Advantageously, the method according to the invention comprises moreover at least one step of recovery of the molecules of interest from the biomass produced. For example, the method according to the invention can comprise moreover at least one step of recovery of the hydrophobic material (which contains lipids and/or pigments) and, optionally, at least one step of extraction of the fatty acids, in particular the EPA and/or the DHA and/or the ARA and/or the ALA, and/or the oleic acid and/or at least one step of extraction of the pigments, in particular the lutein, fucoxanthin, astaxanthin, zeaxanthin, canthaxanthin, echinenone, beta-carotene and phoenicoxanthin from this hydrophobic material.

The recovery of the molecules of interest can be carried out by conventional methods.

In particular, when said molecule of interest is a fatty acid, the method according to the invention optionally comprises, moreover, at least one step of extraction of this fatty acid from said lipids. The methods for the selective extraction of the lipids including EPA, ARA and DHA, are known to one skilled in the art and are, for example, described by [Bligh, E. G. and Dyer, W. J. (1959); A rapid method of total lipid extraction and purification, Can. J. Biochem. Physiol., 37:911-917].

The invention is non-limitatively illustrated by the following examples.

### EXAMPLES

### Example 1

The production of an oil rich in DHA and in canthaxanthin by the strain FCC 1324 of the genus Aurantiochytrium is described below.

Step a):

The cultures of Aurantiochytrium are carried out in 1 to 2 L fermenters (bioreactors) for use with dedicated automatic controllers and computerized supervision. The pH of the system is adjusted by adding base (a 2N sodium hydroxide solution) and/or acid (a 1N sulphuric acid solution). The culture temperature is set to 26° C. Stirring is carried out using 3 stirring rotors mounted on the shaft according to the Rushton configuration (three-blade impellers with downward pumping). The dissolved oxygen pressure is regulated in the medium throughout culture, by the stirring speed (250-600 rpm), the air flow rate (0.25-1 vvm), or the oxygen flow rate (0.1-0.5 vvm). The control parameters, integrated into the automatic supervision system, make it possible to maintain a constant pO₂ at 15%. The bioreactor is equipped with an internal lighting system fixed on the baffles. The intensity as well as the light cycles are controlled by a dedicated automatic device, under computerized supervision. The culture is illuminated with 60 flashes per hour, each flash having a duration of 20 seconds and an intensity of 100 μmol·m⁻²·s⁻¹.

The reactors are inoculated with a preculture prepared on a mixing table (140 rpm) in a temperature-controlled chamber (26° C.) and illuminated between 100 and 200 μE. Pre-cultures and cultures in the bioreactors are carried out in the modified Verduyn medium (sea salts 15 g/L, (NH₄)₂SO₄ 3 g/L, KH₂PO₄ 1 g/L, MgSO₄.7H₂O 0.5 g/L, Na₂EDTA 24 mg/L, ZnSO₄.7H₂O 3 mg/L, MnCl₂.2H₂O 3 mg/L, Na₂MoO₄.2H₂O 0.04 mg/L, FeSO₄.7H₂O 10 mg/L, pantothenate 3.2 mg/L, thiamine hydrochloride 9.5 mg/L, vitamin B12 0.15 mg/L). The carbon-containing substrate used is glucose at concentrations between 60 and 200 g/L.

Continuous feeding with fresh medium is carried out with a dilution level of approximately 0.08 to 0.15 h⁻¹ and with a medium concentrated between 10 and 15 times the initial concentration of each element. One skilled in the art will be able to determine how to implement the continuous culture and calculate the feed flow rate, as well as to determine when the steady state is attained.

Once the steady state is attained, the continuous draw-off from this fermenter can serve to feed the maturation fermenter of step b).

Step b):

The cultures are carried out in 10 to 20 L fermenters (bioreactors) used with dedicated automatic controllers and computerized supervision. The regulation systems as well as the parameterization of the latter are, in every respect, similar to those of step a).

The reactors are inoculated with approximately 50% of the total culture volume of the draw-off from step a); and at the same time, the tank is also fed at the same flow rate with a medium concentrated twice, and which makes it possible to obtain the following final composition: sea salts 15 g/L; glucose between 60 and 120 g/L; (NH₄)₂SO₄ at 0.8 gL; KH₂PO₄ 1 g/L; MgSO₄.7H₂0, 0.5 g/L, Na₂EDTA 24 mg/L, ZnSO₄.7H₂O 3 mg/L, MnCl₂.2H₂O 3 mg/L, Na₂MoO₄.2H₂O 0.04 mg/L, FeSO4.7H₂O 10 mg/L, pantothenate 3.2 mg/L, thiamine hydrochloride of 9.5 mg/L, vitamin B12 0.15 mg/L.

The culture is illuminated with 60 flashes per hour, each flash having a duration of 20 seconds and an intensity of 500 μmol·m⁻²·s⁻¹.

### Monitoring of the Cultures:

The concentration of total biomass is monitored by measuring the dry mass (filtration on a Whatman GF/F filter, then oven drying, at 105° C., for min. 24 h before weighing).

Regarding the quantification of the total lipids, about 10⁸ cells/mL were extracted. Methods for extracting lipids are known to one skilled in the art.

For the quantification of the carotenoids and notably canthaxanthin, 10⁸ cells/mL were extracted. Methods for extraction and analysis of the carotenoids, including canthaxanthin, are known to one skilled in the art.

TABLE 4
Results (n = 3)

| | Total lipids | | |
|---|---|---|---|
| Dry Mass | (% of the | | Canthaxanthin |
| (g/L) | DM) | % DHA | (mg/g of DM) |
| 155 +/− 3.3 | 55 +/− 2.8 | 18.0 +/− 3.0 | 2.1 +/− 0.3 |

Table 4 represents the comparative data for the production of oil rich in DHA by Aurantiochytrium according to the method of the invention (uncoupled) and discontinuous, semi-continuous and continuous methods. The values cited for the discontinuous, semi-continuous and continuous methods are, by way of prediction over one year, based on our previous experiments with this strain and the other similar strains in discontinuous, semi-continuous and continuous culture.

TABLE 5

| UNITARY | | FED- | | |
|---|---|---|---|---|
| OPERATION | BATCH | BATCH | CONTINUOUS | UNCOUPLED |
| Biomass | 5.8% | 29% | 84.3% | 100% |
| produced | | | | |
| (relative | | | | |
| percentage | | | | |
| with respect to | | | | |
| the biomass | | | | |
| produced in | | | | |
| uncoupled | | | | |
| mode) | | | | |
| Effluents | 1,280 | — | 9,690 m³/year | — |
| during | m³/year | | 29.4 m³/day | |
| concentration | 3.9 m³/day | | | |
| Effluents | 280 tonnes/ | 1,400 | 3,990 tonnes/ | 4,810 tonnes/ |
| during drying | year | tonnes/ | year | year |
| (water) | 0.85 | year | 13.3 | 14.6 |
| | tonnes/day | 4.2 | tonnes/day | tonnes/day |
| | | tonnes/ | | |
| | | day | | |
| Ratio Kg | 39 L | 7 L | 24 L | 7 L |
| effluents/Kg | | | | |
| of biomass | | | | |
| produced | | | | |
| Except for | | | | |
| drying | | | | |

Example 2

The continuous production of a biomass with a high cell density and low in fatty matter (5 to 10%) then the accumulation of fatty acids by maturation of this biomass.

Step a): Continuous Culture for the Production of Biomass

The cultures of Aurantiochytrium are carried out in 1 to 2 L reactors for use with dedicated automatic controllers and computerized supervision. The pH of the system is adjusted by adding base (a 2N sodium hydroxide solution) and/or acid (a 1N sulphuric acid solution). The culture temperature is set to 26° C. Stirring is carried out using 3 stirring rotors: 1 Rushton turbine with 6 straight blades positioned at the lower end of the stirring shaft above the sparger and 2 three-blade propellers placed on the stirring shaft (distance between the stirring rotors=1.5 ×the diameter of a propeller).

The dissolved oxygen pressure is regulated in the medium throughout the culture, by the speed of rotation of the stirring shaft (250-600 rpm), the ventilation with air (0.25-1 vvm) or with oxygen (0.1-0.5 vvm). The regulation parameters, incorporated in the automatic supervision controller, make it possible to maintain a constant pO2 at 15%.

The fermenter is equipped with an internal light system fixed to the baffles.

The intensity and the frequency of the illumination cycles are controlled by a dedicated automatic controller with computerized supervision. The culture is illuminated with 60 flashes per hour, each flash having a duration of 20 seconds and an intensity of 100 μmol·m-2·s-1.

The pre-culture is carried out on a mixing table (140 rpm) in a temperature-controlled chamber (26° C.) and illuminated between 100 and 200 μE. The reactor is inoculated at 1% from the pre-culture and operated in discontinuous mode.

Once the substrate contained in the initial medium is consumed and the desired cell density is attained, the draw-off of the culture medium, on the one hand, and the supply of feed solution, on the other hand, are started. The steady state is attained after at least 5 residence times. Once the steady state has stabilized, the operating parameters (ventilation flow rate, power consumption, feed flow rate, pH, volume of mash), the residual concentration of substrate in the mash, as well as the macromolecular concentration and composition of the biomass produced are constant.

For the strain FCC 1324, the composition of the feed solution chosen and the dilution level selected make it possible to produce a volume of mash equivalent to 2.4 times the volume of the fermenter each day.

The regime is considered stable at the end of 50 hours of culture. Once the regime has stabilized, the residual glucose concentration in the mash stabilises around 0 g/L, the cell density stabilizes around 3×10⁹ cells/mL, which corresponds to approximately 65 g/L of biomass, the content of fatty matter in the biomass stabilizes around 5% (gFatty Matter/gDry Matter). The fermentation in continuous mode has been maintained for a total duration of 890 hours, 815 hours of which are under stabilized conditions.

For this example the level of dilution with the feed solution is set at half of the maximum growth rate of the strain on the medium used and under the culture conditions used.

The draw-off flow rate of the medium is adapted in order to take account of the flow rate of the feed solution but also the volume of liquid for correcting the pH.

The composition of the culture medium for starting the culture in batch mode is detailed in Table 1.

The feed solution of the continuous culture contains 15 g/L of commercial sea salt (e.g. INSTANT OCEAN salts), 110 g/L of glucose, and contains concentrations of trace elements, macronutrients and vitamins equivalent to approximately 3 times those contained in the medium used for the pre-culture and the discontinuous culture. Other compositions for the feed solution are possible, as detailed in Table 1.

The concentration of total biomass is monitored by measurement of the dry mass (filtration on GF/F, Whatman, filter then oven drying at 105° C., for a minimum of 24 h before weighing).

Regarding the quantification of the total lipids, approximately 10×⁸ cells were extracted. The methods for the extraction of the lipids are known to one skilled in the art.

For the quantification of the carotenoids, and in particular canthaxanthin, 10×⁸ cells were extracted. Methods for extraction and analysis of the carotenoids, including canthaxanthin, are known to one skilled in the art.

TABLE 6
Characteristics of the steady state obtained in continuous culture.

| | | |
|---|---|---|
| Residual glucose | 0.1 +/− 0.02 | g/L |
| Cell density | 3.109 +/− 0.5.109 | cells/mL |
| Dry Mass | 65 +/− 5 | g/L |
| Fatty acid content | 7% +/− 3% | ok |

Step b) Maturation

The mash produced in continuous culture therefore has a high cell density of a biomass with a very low lipid content (5 to 10%). This biomass is transferred into a maturation tank under culture conditions where it very rapidly accumulates fatty acids (65 G/L in 24 hours). This allows a high concentration of biomass rich in fatty acid to be produced.

In this example, several maturation tests are carried out starting from the mash resulting from the fermenter operated in chemostat mode. The cultures are carried out in 3 to 5 L fermenters (bioreactors) for use with dedicated automatic controllers and computerized supervision. The regulation systems as well as the settings of the latter are, in every respect, similar to those in step a).

In the case of the maturation tests described in this example, the volume taken from the continuous culture is taken in one go. In the case of an industrial fermentation, this transfer is carried out progressively by drawing-off the culture medium.

In the maturation tank, the nutritive solution with a C/N/P ratio of 530:11:1 is progressively added to the volume taken.

The nutritive solution is added into the maturation tank each time that the carbon source (in this case glucose) is exhausted.

The monitoring of the discontinuous culture supplied, carried out in the maturation tank, is carried out according to protocol which is in every respect identical to that of step a). The results are given in Table 7.

TABLE 7
Dry matter, fatty acids and DHA produced in 24 hours and in 48 hours
during the maturation at 25° C. and at a pH of 6.5 (5 repetitions)

| | 24 Hours of culture | 48 Hours of culture |
|---|---|---|
| Dry Matter | 160 g/L (+/−2 g/l) | 185 g/l (+/−5 g/l) |
| Fatty acids | 67 g/L (+/−2 g/l) | 85 g/l (+/−5 g/l) |
| DHA | 16.5 g/L (+/−0.5 g/l) | 21 g/L (+/−2 g/l) |
| Astaxanthin | 0.05 mg/g DM (+/−0.01) | 0.07 mg/g DM (+/−0.01) |

Example 3

The production of biomass that may be used in pisciculture or for the production of lutein by the strain FCC 1520 of the genus Chlorella is described below.

Step a):

The cultures of Chlorella are carried out in 1 to 2 L fermenters (bioreactors) for use with dedicated automatic controllers and computerized supervision. The pH of the system is adjusted by adding base (a 2N sodium hydroxide solution) and/or acid (a 1N sulphuric acid solution). The culture temperature is set to 26° C. Stirring is carried out using 3 stirring rotors mounted on the shaft according to the Rushton configuration (three-blade impellers with downward pumping). The dissolved oxygen pressure is regulated in the medium throughout culture, by the stirring speed (250-600 rpm), the air flow rate (0.25-1 vvm), or the oxygen flow rate (0.1-0.5 vvm). The control parameters, integrated into the automatic supervision system, make it possible to maintain a constant pO₂ at 15%. The bioreactor is equipped with an internal light system fixed on the baffles. The intensity, as well as the light cycles, are controlled by a dedicated automatic device, under computerized supervision. The culture is illuminated with 30 flashes per hour, each flash having a duration of 60 seconds and an intensity of 50 to 100 μmol·m⁻²·s⁻¹.

The reactors are inoculated with a preculture prepared on a mixing table (140 rpm) in a temperature-controlled chamber (26° C.) and illuminated between 100 and 200 μE. Pre-cultures and cultures in the bioreactors are carried out in the following medium: Glucose 20 g/L; KNO3 2 g/L; NaH2PO4 0.54 g/L; Na2HPO4.12H2O 0.179 g/L; MgSO4.7H2O 0.2465 g/L; CaCl2.2H2O 0.0147 g/L; Yeast extract 0.25 g/L; FeSO4.7H2O 0.01035 g/L; H3BO3 0.000061 g/L; MnSO4,H2O 0.000169 g/L; ZnSO4.7H2O 0.000287 g/L; CuSO4.5H2O 0.0000025 g/L; (NH4)6MoO024.4H2O 0.0000125 g/L; Thiamine Hcl (vitamin B1) 0.2 mg/L; Biotin (Vitamin H) 0.001 mg/L; Cyanocobalamin (vitamin B12) 0.001 mg/L.

Continuous feeding with fresh medium is carried out with a dilution level of approximately 0.08 to 0.15 h⁻¹ and with the following medium: Glucose 224.55 g/L; KNO3 22.45 g/L; MgSO4.7H2O 0.165 g/L; CaCl2.2H2O 2.77 g/L; FeSO4.7H2O 0.17 g/L; H3BO3 0.0015 g/L; MnSO4,H2O 0.0042 g/L; ZnSO4.7H2O 0.0072 g/L; CuSO4.5H2O 0.0000625 g/L; (NH4)6MoO024.4H2O 0.0003125 g/L; Thiamine Hcl (vitamin B1) 2.24 mg/L; Biotin (Vitamin H) 0.0112 mg/L; Cyanocobalamin (vitamin B12) 0.0112 mg/L. One skilled in the art will be able to determine how to implement the continuous culture and calculate the feed flow rate, as well as to determine when the steady state is attained.

Once the steady state is attained, the continuous draw-off from this fermenter can serve to feed the maturation fermenter of step b).

Step b):

The cultures are carried out in 10 to 20 L fermenters (bioreactors) used with dedicated automatic controllers and computerized supervision. The regulation systems as well as the settings of the latter are, in every respect, similar to those of step a).

The reactors are inoculated with approximately 50% of the total culture volume with the draw-off from step a); and at the same time, the tank is also fed at the same flow rate with a medium concentrated twice, which makes it possible to obtain the following final composition: Glucose 500 g/L; KNO3 50 g/L; NaH2PO4 13.5 g/L; Na2HPO4. 12H2O 0 4.47 g/L;MgSO4.7H2O 6.1625 g/L; CaCl2.2H2O 0.3675 g/L; FeSO4.7H2O 0.086 g/L; H3BO3 0.061 g/L; MnSO4,H2O 0.169 g/L; ZnSO4.7H2O 0.287 g/L; CuSO4.5H2O 0.0025 g/L; (NH4)6MoO24.4H2O 0.0125 g/L; Thiamine Hcl (vitamin B1) 5 mg/L; Biotin (Vitamin H) 0.025 mg/L; Cyanocobalamin (vitamin B12) 0.025 mg/L.

The culture is illuminated with 30 flashes per hour, each flash having a duration of 20 seconds and an intensity of 100 μmol·m⁻²·s⁻¹.

### Monitoring of the Cultures:

The concentration of total biomass is monitored by measuring the dry mass (filtration on a Whatman GF/F filter, then oven drying, at 105° C., for min. 24 h before weighing).

For the quantification of the carotenoids and notably lutein, 10⁸ cells/mL were extracted. Methods for extraction and analysis of the carotenoids, including lutein, are known to one skilled in the art.

TABLE 8
Results (n = 3)

| Dry Mass | Canthaxanthin |
|---|---|
| (g/L) | (mg/g of DM) |
| 130 +/− 5 | 5.1 +/− 0.1 |

1-23. (canceled)

24. Method for the production of biomass, comprising:
a) the culture of the cells in continuous mode under mixotrophic or heterotrophic conditions in a fermenter, then
b) the continuous and successively feeding of n fermenters operating in semi-continuous mode, n being an integer equal to or greater than 2, with the cells produced in step a) and their culture under mixotrophic conditions
wherein the culture of the cells under mixotrophic conditions is carried out under conditions of illumination that is discontinuous and/or variable over time.

25. Method according to claim 24, wherein the illumination has variations in intensity the amplitude of which is comprised between 5 and 1,000 μmol·m-2·s-1, these variations taking place between 2 and 3,600.

26. Method according to claim 24, wherein the illumination is in the form of flashes.

27. Method according to claim 24, wherein the culture in step a) is carried out in the presence of flashes having an intensity of 50 to 200 μmol·m-2·s-1 and a duration of approximately 1/10th of a second to five minutes, and between 2 and 3,600 flashes per hour.

28. Method according to claim 24, wherein the culture in step b) is carried out in the presence of flashes having an intensity of 50 to 2,000 μmol·m-2·s-1 and a duration of approximately 1/10th of a second to five minutes, and between 2 and 3,600 flashes per hour.

29. Method according to claim 24, wherein the culture in steps a) and/or b) is carried out in the presence of an organic carbon-containing substrate at a concentration from 5 mM to 1.1 M.

30. Method according to claim 24, wherein the cells are eucaryotic or procaryotic unicellular organisms or eucaryotic cells isolated from a multicellular animal, plant or fungal organism, capable of inducing a metabolic activity in response to natural or artificial illumination, under mixotrophic conditions.

31. Method according to claim 24, wherein the eucaryotic or procaryotic unicellular organisms are chosen from marine or fresh-water, photosynthetic or non-photosynthetic protists, yeasts or cyanobacteria.

32. Method according to claim 31, wherein the eucaryotic unicellular organisms are mixotrophic protists chosen from the species of the following genera: Schizochytrium, Thraustochytrium, Odontella, Phaeodactylum, Nanochloris, Crypthecodinium, Monodus, Nannochloropsis, Isochrysis, Euglena, Cyclotella, Nitzschia, Aurantiochytrium, Scenedesmus and/or Tetraselmis.

33. Method according to claim 31, wherein the eucaryotic unicellular organisms are mixotrophic protists chosen from the species Chlorella and Haematococcus.

34. Method according to claim 24, further comprising at least one step of recovery of the biomass resulting from step b).

35. Method according to claim 34, wherein said biomass comprises at least one molecule of interest chosen from an alcohol, an organic acid, a fatty acid, a polysaccharide, a terpene, a pigment, an amino acid, an enzyme, a vitamin, an antibiotic, a compound with pharmacological activity and a pigment.

36. Method according to claim 35, wherein said molecule of interest is chosen from eicosapentaenoic acid (EPA) and docosahexaenoic acid (DHA), arachidonic acid (ARA), α-linolenic acid (ALA), oleic acid, fucoxanthin, canthaxanthin, astaxanthin, lutein and beta-carotene.

37. Method according to claim 24, further comprising at least one step of recovery of the lipids and/or of the pigments.

38. Method according to claim 24, wherein step b) is carried out between 15 and 24° C.

39. Method according to claim 25, wherein the illumination is in the form of flashes.

40. Method according to claim 25, wherein the culture in step a) is carried out in the presence of flashes having an intensity of 50 to 200 μmol·m-2·s-1 and a duration of approximately 1/10th of a second to five minutes, and between 2 and 3,600 flashes per hour.

41. Method according to claim 26, wherein the culture in step a) is carried out in the presence of flashes having an intensity of 50 to 200 μmol·m-2·s-1 and a duration of approximately 1/10th of a second to five minutes, and between 2 and 3,600 flashes per hour.

42. Method according to claim 25, wherein the culture in step b) is carried out in the presence of flashes having an intensity of 50 to 2,000 μmol·m-2·s-1 and a duration of approximately 1/10th of a second to five minutes, and between 2 and 3,600 flashes per hour.

43. Method according to claim 26, wherein the culture in step b) is carried out in the presence of flashes having an intensity of 50 to 2,000 μmol·m-2·s-1 and a duration of approximately 1/10th of a second to five minutes, and between 2 and 3,600 flashes per hour.
